(19) United States
(12) Patent Application Publication
(10) Pub. No.: US 2023/0390400 A1
(43) Pub. Date: Dec. 7, 2023

(54) MACROENCAPSULATION DEVICES

(71) Applicant: Vertex Pharmaceuticals Incorporated, Boston, MA (US)

(72) Inventors: Christopher Thanos, Providence, RI (US); Megan Billings, Warwick, RI (US); Matthew Watson, Stoneham, MA (US)

(73) Assignee: Vertex Pharmaceuticals Incorporated, Boston, MA (US)

(21) Appl. No.: 18/206,274

(22) Filed: Jun. 6, 2023

Related U.S. Application Data
(60) Provisional application No. 63/349,749, filed on Jun. 7, 2022.

(51) Int. Cl. A61K 47/34; B01D 69/12

(52) U.S. Cl. CPC A61K 47/34; B01D 69/125

(57) ABSTRACT

A method of manufacturing a macroencapsulation device includes aligning one or more membranes of the device with a frame of the device, such that a portion of the one or more membranes overlap a portion of the frame. The method also includes deforming the one or more membranes and thermoplastically deforming the frame to form mechanically interlocked regions of the membrane and the frame.

### CROSS-REFERENCE TO RELATED APPLICATIONS

This application claims priority under 35 U.S.C. § 119(e) to U.S. Provisional Application Ser. No. 63/349,749, filed Jun. 7, 2022, and entitled “MACROENCAPSULATION DEVICES,” which is incorporated by reference in it its entirety for all purposes.

### FIELD

Disclosed embodiments are related to macroencapsulation devices and their methods of manufacture.

### BACKGROUND

Therapeutic devices that deliver biological products can be used to treat metabolic disorders, such as diabetes. The therapeutic devices may be implantable to provide a biological product, such as insulin, for an extended period of time. Some of these devices include macroencapsulation devices used to house cells to produce the desired biological product, a matrix including the cells, or other desired therapeutics within.

### SUMMARY

In some embodiments, a method of manufacturing a macroencapsulation device may comprise aligning one or more membranes of the macroencapsulation device with a frame of the macroencapsulation device such that a portion of the one or more membranes may at least partially overlap with a portion of the frame. The method may further comprise deforming the portion of the one or more membranes and thermoplastically deforming the portion of the frame to form a plurality of mechanically interlocked regions of the one or more membranes and the frame.

In other embodiments, a macroencapsulation device may comprise one or more membranes including a sealed interior volume configured to encapsulate a population of cells. The device may also comprise a frame. The one or more membranes may be disposed on the frame. The device may further comprise a plurality of mechanically interlocked regions of the one or more membranes and the frame extending around at least a portion of a perimeter of the one or more membranes.

In further embodiments, a bonding apparatus for manufacturing a macroencapsulation device may comprise a retention mechanism configured to selectively retain a frame of the macroencapsulation device and one or more membranes of the macroencapsulation device in an overlapped configuration with at least a portion of the one or more membranes overlapping at least a portion of the frame. The bonding apparatus may further comprise a heater configured to heat at least the portion of the frame. The bonding apparatus may also include one or more dies configured to deform the portion of the one or more membranes and thermoplastically deform the portion of the frame to form a plurality of mechanically interlocked regions of the one or more membranes and the frame extending around at least a portion of a perimeter of the one or more membranes.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is a top view of a membrane of a macroencapsulation device prior to mounting to a frame, according to one embodiment;

FIG. 1B is a cross-sectional side view of an embodiment of a membrane of a macroencapsulation device, for example as taken along line 1B-1B of FIG. 1A;

FIG. 1C is a cross-sectional side view of an embodiment of a portion of a membrane of a macroencapsulation device in a filled configuration, for example as taken along line 1B-1B of FIG. 1A;

FIG. 1D is a top view of a frame of a macroencapsulation device prior to mounting of one or more associated membranes, according to one embodiment;

FIG. 1E is a close-up top view of a fill port of a macroencapsulation device prior to mounting of one or more associated membranes, according to one embodiment;

FIG. 1F is a cross-sectional side view of a frame of a macroencapsulation device, for example as taken along line 1F-1F of FIG. 1D;

FIG. 2A is a top view of a membrane of a macroencapsulation device mounted to a frame using an adhesive;

FIG. 2B is a top view of a membrane of a macroencapsulation device mounted to a frame using a mechanical bonding technique as disclosed herein, according to one embodiment;

FIG. 3A is a close-up top view of a frame/membrane interface of a macroencapsulation device according to one embodiment where the membrane was attached to a frame using adhesive and was subsequently removed;

FIG. 3B is a close-up top view of a frame/membrane interface of a macroencapsulation device according to one embodiment, where the membrane was attached to a frame using a mechanical bonding technique as disclosed herein and was subsequently removed;

FIG. 4A is a schematic view showing one embodiment of a process for producing a mechanical bond between a frame and a membrane at a first step in producing the bond;

FIG. 4B is a schematic view showing the process of FIG. 4A at a second step in producing the bond;

FIG. 4C is a schematic view showing the process of FIG. 4A at a third step in producing the bond;

FIG. 4D is a schematic view showing the process of FIG. 4A at a fourth step in producing the bond;

FIG. 4E is a schematic view showing the process of FIG. 4A at a fifth step in producing the bond;

FIG. 4F is a schematic view showing the mechanical bond produced by the process of FIGS. 4A-4E;

FIG. 5A is a schematic view of a bonding apparatus for manufacturing a macroencapsulation device, according to one embodiment;

FIG. 5B is a schematic view of a bonding apparatus for manufacturing a macroencapsulation device, according to another embodiment;

FIG. 5C is a schematic view of a bonding apparatus for manufacturing a macroencapsulation device, according to a further embodiment;

FIG. 6 is a schematic view of a bonding apparatus for manufacturing a macroencapsulation device, according to another embodiment;

FIG. 7A is a schematic perspective view of a first die of a bonding apparatus for manufacturing a macroencapsulation device, according to one embodiment;

FIG. 7B is a schematic perspective view of a second die of a bonding apparatus for manufacturing a macroencapsulation device, according to another embodiment;

FIG. 8 is a flow diagram depicting a method of manufacturing a macroencapsulation device, according to one embodiment;

FIG. 9 is a bar graph showing results of cell viability tests performed on both adhesively bonded and mechanically bonded macroencapsulation devices, according to some embodiments;

FIG. 10 is a bar graph showing results of fatigue tests performed on both adhesively bonded and mechanically bonded macroencapsulation devices, according to some embodiments;

FIG. 11A is an image of one embodiment of an adhesively bonded macroencapsulation device after fatigue testing;

FIG. 11B is an image of one embodiment of a mechanically bonded macroencapsulation device after fatigue testing; and

FIG. 12 is a chart showing results of macrophage polarization assays performed on both adhesively bonded and mechanically bonded macroencapsulation devices, according to some embodiments.

### DETAILED DESCRIPTION

Driven by a rising need to deliver biological products to treat various disorders, such as diabetes, different types of implantable therapeutic devices have been engineered. However, typical methods of making such devices are often cumbersome, inefficient, and hard to control. For instance, there is often a lack of precision and control in forming specific structural features associated with the device (e.g., mounting a membrane to a frame using adhesives). In addition, it is oftentimes difficult to precisely form these devices within accepted tolerances to prevent mechanical failure of these devices once implanted.

Several difficulties associated with the use of adhesives in mounting a membrane to a frame of a macroencapsulation device have been recognized. First, such processes may require significant manual operations or interventions. These manual processes may introduce undesirable variability and may reduce efficiencies in manufacturing. Additionally, imperfections and nonuniformities during application of the adhesive may lead to stress concentrations in the membrane, resulting in premature fatigue failure and rupture of the membrane. For example, the viscosity of the adhesive, uneven adhesive application, the contact angle at the point of application, and the porosity of the membrane may lead to variability in the mounting of a membrane to a frame. Adhesives may also be absorbed into the pores of a membrane during application which may compromise the bond between the frame and the membrane in addition to modifying the material properties of the membrane in the area where absorption has occurred.

In view of the above, the inventors have recognized and appreciated the benefits of creating a mechanical bond between one or more membranes and a frame of a macroencapsulation device. In some embodiments, this may be accomplished by raising the temperature of one or more portions of the frame to a point where the frame may be thermoplastically deformed (e.g., above a glass transition, or optionally a melting, temperature of a material of the frame), while maintaining the temperature of the membrane below a melting temperature, and in some embodiments a sintering temperature, of the membrane material. It will be appreciated that thermoplastically deforming a frame and/or a membrane of a macroencapsulation device may include the application of both heat and pressure to accomplish a desired deformation. For example, heat may be applied to the frame and/or membranes to provide the noted temperatures above while a pressure is applied to one or more portions of the membrane and frame that are to be deformed and bonded with one another. Specifically, due to the membrane being flexible, even when below the melting temperature of the membrane, the one or more softened portions of the frame and corresponding portions of the membrane may be deformed when a pressure is applied to form one or more mechanical bonds between the frame and membrane. For example, thermoplastic deformation of the frame and corresponding deformation of the compliant membrane may be used to mechanically interlock the one or more deformed portions of the frame and membrane together. Such techniques may allow a strong and/or durable bond to form at an interface between the frame and membrane. In some instances, the bonded portions of the frame and membrane may extend at least partially around a perimeter of the one or more membranes.

In one specific embodiment, a method of manufacturing a macroencapsulation device may comprise aligning one or more membranes of the macroencapsulation device with a frame of the macroencapsulation device such that a portion of the one or more membranes at least partially overlaps a portion of the frame. The method may further comprise deforming the portion of the one or more membranes and thermoplastically deforming the portion of the frame to form a plurality of mechanically interlocked regions of the one or more membranes and the frame. In some embodiments, the frame and/or the membrane may be heated. For example, the frame may be heated to a temperature that is below a melting temperature or sintering temperature of the membrane(s) in order to facilitate thermoplastic deformation of the frame. In some embodiments, the deformation may be carried out in multiple steps, including a first deformation and a second deformation. In other embodiments, the deformation may be carried out in a single step. The plurality of mechanically interlocked regions may extend around a portion of an outer perimeter of the one or more membranes, and in some embodiments, at least partially around a sealed interior volume disposed between two opposing layers of the one or more membranes.

As will be described in more detail below, some embodiments of a macroencapsulation device may include one or more membranes comprising a biocompatible polymer such as polytetrafluoroethylene (PTFE) or expanded polytetrafluoroethylene (ePTFE). Some embodiments may include a frame comprising a thermoplastic material, such as polyetheretherketone (PEEK). In certain embodiments, a macroencapsulation device may include a frame formed from PEEK and one or more membranes formed from ePTFE, although the disclosure is not limited to these embodiments or to any particular combination of the materials described herein (and equivalents thereof). It will be appreciated that in the context of a macroencapsulation device, there may be significant challenges in creating a mechanical bond between a frame comprising a thermoplastic such as PEEK, and a membrane comprising a polymer such as ePTFE due to difficulties in welding these materials. For example, such a bonding process may result in significant heat and/or pressure being applied to the interface between the frame and the membrane. However, application of excessive heat or pressure to polymer membranes may drastically impact the porosity, permeability, or other material properties of the membrane, which may impact the functionality of the entire device. In this respect, forming mechanically interlocked regions of the frame and membrane, rather than simply forming a typical weld, may be desirable to facilitate a mechanical bond in difficult to bond materials though the disclosed methods and devices may be used with any appropriate combination of frame and membrane materials as the disclosure is not so limited.

Macroencapsulation devices manufactured using the disclosed systems and processes may offer a number of benefits. For example, the disclosed systems and processes may allow for easier control over the distribution of membrane slack relative to a supporting frame due to the interlocking regions taking up and distributing the membrane slack. The disclosed bonding techniques may also improve bonding uniformity and strength as compared to a typical adhesive based bond. This may contribute to improvements in a fatigue life of a macroencapsulation device as well. In some embodiments, mechanically interlocked regions may create a strong and/or durable bond such that the membrane(s) may become likely to experience fatigue failure or other mechanical failure before the bond fails, thereby mitigating the bond as a potential mechanical failure point in the device.

In some embodiments, the macroencapsulation device does not include an adhesive or the use of an adhesive-based bond. In some embodiments, the elimination or reduction of adhesive may reduce an immune response in a patient during implantation and/or residence of the macroencapsulation device. For example, an inflammatory response may be reduced by a reduction in the amount of adhesive used. Additionally or alternatively, a reduction in adhesive may improve cell viability and/or reduce cytotoxicity in the tissue surrounding an implanted device. In some embodiments, the device does not include an adhesive. In some embodiments, the present disclosure may include biocompatible adhesives at any appropriate location of the device. Thus, in some embodiments, the device comprises both mechanically interlocking regions and a biocompatible adhesive. For example, some devices may include a combination of adhesives and mechanically interlocking regions at the interface between the membrane(s) and the frame.

In addition to the above, distribution of slack may be facilitated by one or more manufacturing fixtures during manufacture of a macroencapsulation device. In some embodiments, a bonding apparatus for manufacturing the macroencapsulation device may include geometric features configured to facilitate distribution of slack in a membrane. For example, in certain embodiments, a die of the bonding apparatus may include geometric features such as grooves, ridges, and/or crenellations that are configured to uniformly distribute the excess surface area of the one or more membranes of a macroencapsulation device around the bonding area of a corresponding frame the one or more membranes are to be bonded with. In one such embodiment, the groves, ridges, and/or crenelations may be uniformly distributed around a perimeter of a dome or other portion of a bonding apparatus contacting the one or more membranes during a bonding process. Additionally, the grooves, ridges, and/or crenelations may extend radially outward from a central portion of the dome or other portion of the bonding apparatus contacting the one or more membranes during the bonding process. Such geometric features may increase a surface area of the die, allowing slack to be distributed across the surface area in a controlled manner when the membrane is conformed to the surface of the die (for example, using suction as will be described below).

A macroencapsulation device may include multiple layers of membranes. At least one exterior membrane of these multiple layers of membranes may be semipermeable. However, embodiments in which each of the membranes is semipermeable or where at least one of the membranes within a device are substantially impermeable are also contemplated. Further, a device may include two stacked membranes, three stacked membranes, and/or any other appropriate number of membranes as the disclosure is not limited in this fashion. For example, in one embodiment including two membranes, either membrane may be semipermeable and the other impermeable or both may be semipermeable. Accordingly, it should be understood that the current disclosure is not limited to any particular combination of membranes within a stacked structure.

In some embodiments, a macroencapsulation device may include at least one population of cells disposed within an internal volume of the device. For example, the population of cells may be disposed within an internal volume formed between two or more opposing layers of one or more exterior membranes of the device where an exterior edge of the internal volume may be defined by one or more bonds extended around at least a portion, and in some instances an entire, perimeter of the membranes or other appropriate portion of the membranes. In such an embodiment, at least the exterior membranes of the device may be configured to block passage of the one or more populations of cells out of the device. Accordingly, the one or more populations of cells may be retained within the interior volume of the device. While the use of two exterior membranes forming a single internal volume is primarily described, the use of multiple intermediate membranes positioned between the exterior membranes of a device and/or multiple unconnected interior volumes within a device are also contemplated. Additionally, instances in which a single membrane is folded over and bonded to itself to provide two opposing membranes to form the interior volume are also contemplated.

Although expanded polytetrafluoroethylene (ePTFE) is primarily described herein for use as a membrane material, the membranes of a macroencapsulation device may be formed from any appropriate biocompatible material. The biocompatible material may be substantially inert towards cells housed within the macroencapsulation device and the surrounding tissue. The biocompatible material may comprise a synthetic polymer or a naturally occurring polymer. In some embodiments, the polymer may also be a linear polymer, a cross linked polymer, a network polymer, an addition polymer, a condensation polymer, an elastomer, a fibrous polymer, a thermoplastic polymer, a non-degradable polymer, combinations of the foregoing, and/or any other appropriate type of polymer as the disclosure is not limited in this fashion. As noted above, in one embodiment, a polymer may comprise expanded polytetrafluoroethylene (ePTFE). Appropriate types of polymers may also comprise polyvinylchloride (PVC), polyethylene (PE), polypropylene (PP), polymethylmethacrylate (PMMA), polystyrene (PS), polytetrafluoroethylene (PTFE), expanded polytetrafluoroethylene (ePTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly(lactic-co-glycolic acid) (PLGA), poly-L-lactide (PLLA), polyacrylonitrile (PAN), electrospun PAN/PVC, any combination of the foregoing, and/or any other appropriate polymeric material. In some embodiments, a membrane used with any of the embodiments disclosed herein may comprise PVDF. In some embodiments, a membrane used with any of the embodiments disclosed herein may comprise electrospun PAN PVC. In some embodiments, a membrane used with any of the embodiments disclosed herein may comprise PES. In some embodiments, a membrane used with any of the embodiments disclosed herein may comprise PS. In some embodiments, a membrane used with any of the embodiments disclosed herein may comprise PAN. In some embodiments, a membrane used with any of the embodiments disclosed herein may comprise Polycarbonate. In some embodiments, a membrane used with any of the embodiments disclosed herein may comprise polypropylene. In some embodiments, a membrane used with any one of the embodiments disclosed herein may comprise PVC. In some embodiments, a membrane used with any one of the embodiments disclosed herein may comprise PU. In some embodiments, a membrane used with any one of the embodiments disclosed herein may comprise PET. In some embodiments, a membrane used with any one of the embodiments disclosed herein may comprise PCL. In some embodiments, a membrane used with any one of the embodiments disclosed herein may comprise PLGA. In some embodiments, a membrane used with any one of the embodiments disclosed herein may comprise PLLA. In some embodiments, a membrane used with any one of the embodiments disclosed herein may comprise PMMA. In some embodiments, a membrane used with any one of the embodiments disclosed herein may comprise PEI. In some embodiments, a membrane used with any one of the embodiments disclosed herein may comprise nylon. In some embodiments, a membrane used with any one of the embodiments disclosed herein may comprise PTFE. In some embodiments, a membrane used with any one of the embodiments disclosed herein may comprise PE. The synthesis methods used for forming one or more of the porous membranes from the above noted polymeric materials may include, but are not limited to, expansion, solvent-casting, immersion precipitation and phase separation, electrospinning, methods that yield isoreticular networks, methods that yield trabecular networks, or any other appropriate method of forming a porous polymer membrane.

Sintering of a membrane may be used to alter the porosity and flux properties of a membrane. For example, the sintering may increase the porosity of the membrane while maintaining its pore structure. The sintering may also improve the mechanical stability and diffusive flux of the membrane. In some instances, a sintered membrane can have a lower melting temperature than an unsintered membrane of the same type. Further, sintered membranes may exhibit a different energy release during a differential scanning calorimetry scan, indicating a more relaxed structure in addition to the thickened porous network exhibited in sintered materials.

In view of the above, sintering may be used to alter the porosity and/or mechanical properties of the membranes, which in turn can be used to tune the porosity and the flux properties of the macroencapsulation device. Accordingly, in some embodiments, any desired combination of sintered and/or unsintered membranes or membrane layers may be used. For instance, two exterior membrane layers of a device may be bonded together where either a sintered and unsintered membrane are bonded together, two sintered membranes are bonded together, or two unsintered membranes are bonded together. Further, any number of intermediate membranes positioned between these exterior membranes may be used where these intermediate membranes may be sintered or unsintered.

The membranes of a macroencapsulation device as described herein may be made from porous membrane materials that are configured to allow for transport through the membranes of materials, such as a biological product, with a molecular weight less than about 3000 kDa, 2000 kDa, 1000 kDa, 500 kDa, 400 kDa, 300 kDa, 200 kDa, 100 kDa, 50 kDa, 40 kDa, 30 kDa, 20 kDa, 10 kDa, 6 kDa, 5 kDa, 4 kDa, 3 kDa, 2 kDa, 1 kDa, and/or any other appropriate range of molecular weights depending on the desired application. The membranes of a macroencapsulation device as described herein may be made from porous membrane materials that are configured to allow for transport through the membranes of materials, such as a biological product, only within the molecular weight range of 1-3000 kDa, 1-2000 kDa, 1-1000 kDa, 1-500 kDa, 1-400 kDa, 1-300 kDa, 1-200 kDa, 1-100 kDa, 1-50 kDa, 1-40 kDa, 1-30 kDa, 1-20 kDa, 1-10 kDa, 1-6 kDa, 1-5 kDa, 1-4 kDa, 1-3 kDa, or 1-2 kDa. For example, the one or more membranes of a macroencapsulation device may be configured to permit the flow of insulin through the membranes which has a molecular weight of about 5.8 kDa. In some embodiments, the one or more membranes of a macroencapsulation device may be configured to permit the flow of materials, such as a biological product, only within the range of 1-10 kDa. In some embodiments, the one or more membranes of a macroencapsulation device may be configured to permit the flow of materials, such as a biological product, only within the range of 1-6 kDa. In some embodiments, the one or more membranes of a macroencapsulation device may be configured to permit the flow of materials, such as a biological product, only within the range of 1-5 kDa. In some embodiments, the one or more membranes of a macroencapsulation device may be configured to permit the flow of materials, such as a biological product, only within the range of 1-4 kDa. In some embodiments, the one or more membranes of a macroencapsulation device may be configured to permit the flow of materials, such as a biological product, only within the range of 1-3 kDa. In some embodiments, the one or more membranes of a macroencapsulation device may be configured to permit the flow of materials, such as a biological product, only within the range of 1-2 kDa.

To provide the desired selectivity, the porous membranes used with the macroencapsulation devices disclosed herein may have an open porous structure with average pore sizes that are greater than or equal to about 1 nm, 5 nm, 10 nm, 15 nm, 20 nm, 30 nm, 40 nm, 50 nm, 60 nm, 70 nm, 80 nm, 90 nm, 100 nm, 200 nm, 300 nm, and/or any other appropriate size range. Correspondingly, the average pore size of the various membranes described herein may have an average pore size that is less than or equal to 2500 nm, 2000 nm, 1700 nm, 1500 nm, 1400 nm, 1300 nm, 1200 nm, 1100 nm, 1000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, and/or any other appropriate size range. Combinations of the foregoing are contemplated including, for example, an average pore size that is between or equal to 1 nm and 20 nm, 1 nm and 2500 nm, 50 nm and 1200 nm, and/or any other appropriate combination. In some embodiments, the average pore size of the various membranes described herein is between 25 nm and 1500 nm. In some embodiments, the average pore size of the various membranes described herein is between 50 nm and 1200 nm. In some embodiments, the average pore size of the various membranes described herein is between 50 nm and 1000 nm. In some embodiments, the average pore size has an upper size limit of 1500 nm. In some embodiments, the average pore size has an upper size limit of 1200 nm. In some embodiments, the average pore size has a lower size limit of 25 nm. In some embodiments, the average pore size has a lower size limit of about 50 nm. While specific average pore sizes are described above, it should be understood that any appropriate average pore size may be used for the various membranes described herein including average pore sizes both greater than and less than those noted above.

In some embodiments, charge exclusion properties may be included in the membrane. For example, a surface charge of the membranes may be modulated with external coatings, plasma treatments, or other surface treatments to achieve neutral, positive, negative, or zwitterionic properties based on the isoelectric point of the desired ancillary agent. The agent may be a protein, complexed small molecule, and/or any other appropriate agent depending on the desired application.

To provide sufficient strength and/or rigidity for a macroencapsulation device, the various membranes and frames may be made from materials that are sufficiently stiff. The desired stiffness may be provided via an appropriate combination of a material's Young's modulus (also referred to as an Elastic modulus), thickness, and overall construction which may be balanced with a desired permeability of the device. Appropriate Young's moduli for the various membranes and frames described herein may be at least 10⁵ Pa, 10⁶ Pa, 10⁷ Pa, 10⁸ Pa, 10⁹ Pa, and/or 10¹⁰ Pa. Other appropriate Young's moduli for the various membranes and frames described herein may be used including moduli both greater than and less than these ranges. Ranges between the foregoing Young's moduli are contemplated including, for example, a Young's modulus between or equal to about 10⁶ Pa and 10¹⁰ Pa. In some embodiments, the macroencapsulation device includes at least one hydrophilic membrane.

The frame of a macroencapsulation device may be formed from any appropriate biocompatible thermoplastic material. As previously noted, in some embodiments, an appropriate material for the frame may include polyetheretherketone (PEEK). Appropriate materials for the frame may also include, but are not limited to polycarbonate, polyurethane, polyetheretherketone (PEEK), Polyvinyl Chloride (PVC), poly(oxymethylene), poly(methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), polycaprolactone, poly(lactide), poly(glycolic acid), poly lactide-co-glycolide, ethylene vinyl acetate copolymer, polyamides, poly(butylene) therephthalate, combinations of the foregoing, and/or any other appropriate thermoplastic material. In addition to the use of a thermoplastic material in a frame, embodiments in which a frame includes a thermoplastic portion configured to be bonded to a membrane and another non-thermoplastic portion are also contemplated as the disclosure is not limited to frames made completely from a thermoplastic material. In some embodiments, an appropriate material for the frame includes polypropylene. In some embodiments, an appropriate material for the frame includes fluorinated ethylene propylene (FEP). In some embodiments, an appropriate material for the frame includes ultra-high density polyethylene (UHDPE). In some embodiments, an appropriate material for the frame includes polycarbonate. In some embodiments, an appropriate material for the frame includes polyurethane. In some embodiments, an appropriate material for the frame includes PVC. In some embodiments, an appropriate material for the frame includes poly(oxymethylene). In some embodiments, an appropriate material for the frame includes poly(methyl methacrylate (PMMA). In some embodiments, an appropriate material for the frame includes thermoplastic polymer based composites. In some embodiments, an appropriate material for the frame includes polypropylene. In some embodiments, an appropriate material for the frame includes LDPE. In some embodiments, an appropriate material for the frame includes HDPE. In some embodiments, an appropriate material for the frame includes polycaprolactone. In some embodiments, an appropriate material for the frame includes poly (lactide). In some embodiments, an appropriate material for the frame includes poly(glycolic acid). In some embodiments, an appropriate material for the frame includes poly lactide-co-glycolide. In some embodiments, an appropriate material for the frame includes ethylene vinyl acetate copolymer. In some embodiments, an appropriate material for the frame includes polyamides. In some embodiments, an appropriate material for the frame includes poly(butylene) therephthalate. In other embodiments, an appropriate material for the frame or portion of the frame may include titanium, graphene, stainless steel, or other appropriate biocompatible material exhibiting sufficient rigidity to function as a frame for the macroencapsulation device.

Although the present disclosure relates to methods of bonding one or more membranes to a frame of a macroencapsulation device, it should be understood that the present disclosure is not limited to the use of any particular method for bonding one membrane to another, or for bonding two or more membranes together. The membranes described in the various embodiments of macroencapsulation devices described herein may be bonded to one another using any appropriate bonding method as the disclosure is not limited in this fashion. For example, adjacent membranes may be bonded to one another using an epoxy, a weld, or other fusion based technique (e.g., ultrasonic bonding, laser bonding, physical bonding, thermal bonding, etc.), mechanical clamping using a frame or fixture, and/or any other appropriate bonding method. In one specific embodiment, adjacent membranes may be bonded using a heated tool that is used to press or strike two or more membranes against each other for a set fusion time with a predetermined pressure and/or force.

A macroencapsulation device as described herein may have any appropriate combination of internal volumes, external dimensions, and/or other appropriate physical parameters. For example, an internal volume encompassed by the outer membranes of a macroencapsulation device may be between or equal to 40 μL and 250 μL. A width, or maximum transverse dimension, of the macroencapsulation device may also be between about 20 mm and 80 mm. Additionally, to provide a desired diffusion of oxygen into the interior of a macroencapsulation device to support cells contained therein, a maximum oxygen diffusion distance from an exterior of the device to an interior portion of the device including a population of cells may be less than 50 μm, 100 μm, 150 μm, 200 μm, 250 μm, 300 μm, 350 μm, 400 μm, 450 μm, or 500 μm. In some embodiments, a maximum diffusion distance from an exterior of the device to an interior portion of the device including a population of cells is less than 500 μm. In some embodiments, a maximum diffusion distance from an exterior of the device to an interior portion of the device including a population of cells is less than 450 μm. In some embodiments, a maximum diffusion distance from an exterior of the device to an interior portion of the device including a population of cells is less than 400 μm. In some embodiments, a maximum diffusion distance from an exterior of the device to an interior portion of the device including a population of cells is less than 350 μm. In some embodiments, a maximum diffusion distance from an exterior of the device to an interior portion of the device including a population of cells is less than 300 μm. In some embodiments, a maximum diffusion distance from an exterior of the device to an interior portion of the device including a population of cells is less than 250 μm. In some embodiments, a maximum diffusion distance from an exterior of the device to an interior portion of the device including a population of cells is less than 200 μm. In some embodiments, a maximum diffusion distance from an exterior of the device to an interior portion of the device including a population of cells is less than 150 μm. In some embodiments, a maximum diffusion distance from an exterior of the device to an interior portion of the device including a population of cells is 150 μm. In some embodiments, a maximum diffusion distance from an exterior of the device to an interior portion of the device including a population of cells is between 50 μm and 200 μm. In some embodiments, a maximum diffusion distance from an exterior of the device to an interior portion of the device including a population of cells is between 125 μm and 175 μm.

Correspondingly, a maximum thickness, or dimension perpendicular to a maximum transverse dimension, of the overall device and/or an internal volume located within the device may be less than 25 μm, 50 μm, 100 μm, 150 μm, 200 μm, 250 μm, 300 μm, 350 μm, 400 μm, 450 μm, or 500 μm. In some embodiments, a maximum thickness, or dimension perpendicular to a maximum transverse dimension, of the overall device and/or an internal volume located within the device is between 250 μm and 700 μm. In some embodiments, a maximum thickness, or dimension perpendicular to a maximum transverse dimension, of the overall device and/or an internal volume located within the device is between 250 μm and 500 μm. In some embodiments, a maximum thickness, or dimension perpendicular to a maximum transverse dimension, of the overall device and/or an internal volume located within the device is between 400 μm and 500 μm. In some embodiments, a maximum thickness, or dimension perpendicular to a maximum transverse dimension, of the overall device and/or an internal volume located within the device is less than 500 μm. In some embodiments, a maximum thickness, or dimension perpendicular to a maximum transverse dimension, of the overall device and/or an internal volume located within the device is 500 μm.

In some embodiments, a maximum thickness, or dimension perpendicular to a maximum transverse dimension, of the frame may be less than or equal to 4 mm, 3 mm, 2 mm, 1 mm, or any other appropriate thickness. In some embodiments, a maximum thickness, or dimension perpendicular to a maximum transverse dimension, of the frame may be less than or equal to 4 mm. In some embodiments, a maximum thickness, or dimension perpendicular to a maximum transverse dimension, of the frame may be less than or equal to 3 mm. In some embodiments, a maximum thickness, or dimension perpendicular to a maximum transverse dimension, of the frame may be less than or equal to 2 mm. In some embodiments, a maximum thickness, or dimension perpendicular to a maximum transverse dimension, of the frame may be less than or equal to 1 mm. Additionally or alternatively, in some embodiments, the maximum thickness of the frame may be greater than or equal to 0.5 mm, 1 mm, 1.5 mm, 2 mm, or any other appropriate thickness. In some embodiments, the maximum thickness of the frame may be greater than or equal to 0.5 mm. In some embodiments, the maximum thickness of the frame may be greater than or equal to 1 mm. In some embodiments, the maximum thickness of the frame may be greater than or equal to 1.5 mm. In some embodiments, the maximum thickness of the frame may be greater than or equal to 2 mm. Intermediate values between the foregoing are also contemplated, as are combinations of the foregoing and values greater than and/or less than the foregoing. For example, in some embodiments a maximum frame thickness may be greater than or equal to 0.5 mm and less than or equal to 4 mm. In some embodiments, a maximum frame thickness may be greater than or equal to 1.5 mm and less than or equal to 2.5 mm.

Additionally, in some embodiments, a frame may include a frame/membrane interface region, which may be configured to be bonded with one or more membranes of the device. In some embodiments, the frame/membrane interface region may extend radially inwardly from the frame, and may have a maximum thickness, or dimension perpendicular to a maximum transverse dimension of the device, which may be the same as or different from a maximum thickness of the overall frame. In some embodiments, a maximum interface region thickness may be less than a maximum frame thickness. For example, in some embodiments, a maximum interface region thickness may be less than or equal to 2 mm, 1.5 mm, 1 mm, 0.5 mm, or any other appropriate thickness. In some embodiments, a maximum interface region thickness may be less than or equal to 2 mm. In some embodiments, a maximum interface region thickness may be less than or equal to 1.5 mm. In some embodiments, a maximum interface region thickness may be less than or equal to 1 mm. In some embodiments, a maximum interface region thickness may be less than or equal to 0.5 mm. Additionally or alternatively, in some embodiments, a maximum interface region thickness may be greater than or equal to 0.3 mm, 0.5 mm, 1 mm, 1.5 mm, or any other appropriate thickness. Intermediate values between the foregoing are also contemplated, as are combinations of the foregoing and values greater than and/or less than the foregoing. In some embodiments, a maximum interface region thickness may be greater than or equal to 0.3 mm. In some embodiments, a maximum interface region thickness may be greater than or equal to 0.5 mm. In some embodiments, a maximum interface region thickness may be greater than or equal to 1 mm. In some embodiments, a maximum interface region thickness may be greater than or equal to 1.5 mm. For example, in some embodiments, a maximum interface region thickness may be greater than or equal to 0.3 mm and less than or equal to 2 mm. In some embodiments, a maximum interface region thickness may be greater than or equal to 0.3 mm and less than or equal to 0.5 mm.

As noted above, a frame/membrane interface region may extend from the frame, for example in a radial direction inwardly (e.g., toward a center of the device, frame, and/or membrane(s) thereof), and may have any appropriate width in the radial direction. In some embodiments, a frame/membrane interface region may have a radial interface region width less than or equal to 5 mm, 4 mm, 3 mm, 2 mm, or any other appropriate width. In some embodiments, a frame/membrane interface region may have a radial interface region width less than or equal to 5 mm. In some embodiments, a frame/membrane interface region may have a radial interface region width less than or equal to 4 mm. In some embodiments, a frame/membrane interface region may have a radial interface region width less than or equal to 3 mm. In some embodiments, a frame/membrane interface region may have a radial interface region width less than or equal to 2 mm. Additionally or alternatively, in some embodiments, an interface region width may be greater than or equal to 0.75 mm, 1 mm, 1.5 mm, 2 mm, or any other appropriate width. In some embodiments, an interface region width may be greater than or equal to 0.75 mm. In some embodiments, an interface region width may be greater than or equal to 1 mm. In some embodiments, an interface region width may be greater than or equal to 1.5 mm. In some embodiments, an interface region width may be greater than or equal to 2 mm. Intermediate values between the foregoing are also contemplated, as are combinations of the foregoing and values greater than and/or less than the foregoing. For example, in some embodiments a maximum interface region width may be greater than or equal to 0.75 mm and less than or equal to 5 mm. In some embodiments, a maximum interface region width may be greater than or equal to 1.0 mm and less than or equal to 2.0 mm. Additionally, although radial widths are described herein, it will be appreciated that the widths described above are not limited to extending in a radial direction, as the devices are not limited to a circular geometry. For example, a device having one or more linear sides may include a frame/membrane interface region having a width extending inwardly perpendicular from a linear side.

Further, in some embodiments, an outer surface area to volume ratio of the device may be greater than or equal to about 20 cm⁻¹, 40 cm⁻¹, 50 cm⁻¹, 60 cm⁻¹, 80 cm⁻¹, 100 cm⁻¹, 120 cm⁻¹, 150 cm⁻¹ 200 cm⁻¹, 300 cm⁻¹, 400 cm⁻¹, 500 cm⁻¹, 600 cm⁻¹, 700 cm⁻¹, 800 cm⁻¹, 900 cm⁻¹, or 1000 cm⁻¹. In some embodiments, an outer surface area to volume ratio of the device may be between 25 cm⁻¹ and 1250 cm⁻¹. In some embodiments, an outer surface area to volume ratio of the device may be between 50 cm⁻¹ and 1000 cm⁻¹. In some embodiments, an outer surface area to volume ratio of the device may be between 100 cm⁻¹ and 500 cm⁻¹. Ranges extending between any of the forgoing values for the various dimensions and parameters as well as ranges both greater than and less those noted above are also contemplated.

As described throughout the present disclosure, a plurality of bonded portions may be provided in the membrane(s) of a macroencapsulation device. In some embodiments, each bonded portion may be formed in a rounded or circular shape, each having a bonded diameter. In various embodiments, a bonded diameter may be greater than or equal to 200 μm, 500 μm, 1000 μm, 1500 μm, or any other appropriate distance or diameter. In some embodiments, a bonded diameter may be greater than or equal to 200 μm. In some embodiments, a bonded diameter may be greater than or equal to 500 μm. In some embodiments, a bonded diameter may be greater than or equal to 1000 μm. In some embodiments, a bonded diameter may be greater than or equal to 1500 μm. Additionally or alternatively, in some embodiments, a bonded diameter may be less than or equal to 2000 μm, 1500 μm, 1000 μm, 500 μm, or any other appropriate distance or diameter. In some embodiments, a bonded diameter may be less than or equal to 2000 μm. In some embodiments, a bonded diameter may be less than or equal to 1500 μm. In some embodiments, a bonded diameter may be less than or equal to 1000 μm. In some embodiments, a bonded diameter may be less than or equal to 500 μm. Intermediate values between the foregoing are also contemplated, as are combinations of the foregoing and values greater than and/or less than the foregoing. For example, in some embodiments a bonded diameter may be greater than or equal to 200 μm and less than or equal to 2000 μm. In some embodiments, a bonded diameter may be greater than or equal to 700 μm and less than or equal to 800 μm. Additionally, although bonded diameters are described herein, it will be appreciated that the dimensions of the bonded portions described above are not limited to diameters, as the bonded portions are not limited to a circular geometry. For example, in a bonded portion formed in a rectilinear shape, the dimensions above may apply to a distance between opposing sides of the rectilinear shape.

In some embodiments, it may be desirable to improve the vascularization of a macroencapsulation device. Accordingly, in certain embodiments, one or more through holes may be formed in the one or more bonded portions located within an interior portion of the membranes disposed radially inwards from a frame of the device. These through holes may permit vasculature to growth through the through holes in addition to growing around the upper and lower surfaces of the device. The one or more through holes may be formed in the bonded portions of the membranes using laser ablation, mechanical puncture, cutting, or any other appropriate method of forming a through hole in the one or more bonded portions of the membranes.

In some embodiments, a through hole of a bonded portion may have a diameter, or other maximum transverse dimension perpendicular to an axis extending through the through hole, that is greater than or equal to 100 μm, 125 μm, 150 μm, 200 μm, 300 μm, or any other appropriate through hole diameter. In some embodiments, a through hole of a bonded portion may have a diameter, or other maximum transverse dimension perpendicular to an axis extending through the through hole, that is greater than or equal to 100 μm. In some embodiments, a through hole of a bonded portion may have a diameter, or other maximum transverse dimension perpendicular to an axis extending through the through hole, that is greater than or equal to 125 μm. In some embodiments, a through hole of a bonded portion may have a diameter, or other maximum transverse dimension perpendicular to an axis extending through the through hole, that is greater than or equal to 150 μm. In some embodiments, a through hole of a bonded portion may have a diameter, or other maximum transverse dimension perpendicular to an axis extending through the through hole, that is greater than or equal to 200 μm. In some embodiments, a through hole of a bonded portion may have a diameter, or other maximum transverse dimension perpendicular to an axis extending through the through hole, that is greater than or equal to 250 μm. In some embodiments, a through hole of a bonded portion may have a diameter, or other maximum transverse dimension perpendicular to an axis extending through the through hole, that is greater than or equal to 300 μm. Additionally or alternatively, in some embodiments, a through hole diameter, or other maximum transverse dimension perpendicular to an axis extending through the through hole, may be less than or equal to 700 μm, 650 μm, 600 μm, 500 μm, 300 μm, or any other appropriate through hole diameter. In some embodiments, a through hole diameter, or other maximum transverse dimension perpendicular to an axis extending through the through hole, may be less than or equal to 700 μm. In some embodiments, a through hole diameter, or other maximum transverse dimension perpendicular to an axis extending through the through hole, may be less than or equal to 650 μm. In some embodiments, a through hole diameter, or other maximum transverse dimension perpendicular to an axis extending through the through hole, may be less than or equal to 600 μm. In some embodiments, a through hole diameter, or other maximum transverse dimension perpendicular to an axis extending through the through hole, may be less than or equal to 500 μm. In some embodiments, a through hole diameter, or other maximum transverse dimension perpendicular to an axis extending through the through hole, may be less than or equal to 300 μm. Intermediate values between the foregoing are also contemplated, as are combinations of the foregoing and values greater than or less than the foregoing. For example, in some embodiments a through hole diameter, or other maximum transverse dimension perpendicular to an axis extending through the through hole, may be greater than or equal to 250 μm and less than or equal to 350 μm. In some embodiments, a through hole diameter, or other maximum transverse dimension perpendicular to an axis extending through the through hole, may be greater than or equal to 200 μm and less than or equal to 400 μm.

Additionally, in some embodiments, a macroencapsulation device may be formed with any appropriate distance or spacing between two adjacent bonded portions (referred to herein as a bond spacing). For example, in some embodiments, a bond spacing may be greater than or equal to 1 mm, 2, mm, 3 mm, or any other appropriate spacing. In some embodiments, a bond spacing may be greater than or equal to 1 mm. In some embodiments, a bond spacing may be greater than or equal to 2 mm. In some embodiments, a bond spacing may be greater than or equal to 3 mm. Additionally or alternatively, a bond spacing may be less than or equal to 5 mm, 4 mm, 3 mm, or any other appropriate bond spacing. In some embodiments, a bond spacing may be less than or equal to 5 mm. In some embodiments, a bond spacing may be less than or equal to 4 mm. In some embodiments, a bond spacing may be less than or equal to 3 mm. Intermediate values between the foregoing are also contemplated, as are combinations of the foregoing and values greater than or less than the foregoing. For example, in some embodiments, a bond spacing may be greater than or equal to 1 mm and less than or equal to 5 mm. In some embodiments, a bond spacing may be greater than or equal to 1 mm and less than or equal to 1.5 mm.

Further, in some embodiments, two or more membranes may be bonded together at a bonded perimeter, and the bonded portions may be located within the bonded perimeter. In various embodiments, the bonded perimeter may be formed in any appropriate shape, including any appropriate round, elongated, rectilinear, polygonal (e.g., pentagonal, hexagonal, octagonal, etc.), and/or any other appropriate regular or irregular shape. In some embodiments, a bonded perimeter may be generally formed as a circle having a diameter. In some embodiments, the diameter, or other maximum transverse dimension, of the bonded perimeter may be greater than or equal to 1 cm, 1.5 cm, 2 cm, 2.5 cm, or any other appropriate distance or diameter. In some embodiments, the diameter, or other maximum transverse dimension, of the bonded perimeter may be greater than or equal to 1 cm. In some embodiments, the diameter, or other maximum transverse dimension, of the bonded perimeter may be greater than or equal to 1.5 cm. In some embodiments, the diameter, or other maximum transverse dimension, of the bonded perimeter may be greater than or equal to 2 cm. In some embodiments, the diameter, or other maximum transverse dimension, of the bonded perimeter may be greater than or equal to 2.5 cm. Additionally or alternatively, in some embodiments, the diameter or other maximum transverse dimension of the bonded perimeter may be less than or equal to 7 cm, 6.5 cm, 6 cm, 5 cm, or any other appropriate distance or diameter. In some embodiments, the diameter or other maximum transverse dimension of the bonded perimeter may be less than or equal to 7 cm. In some embodiments, the diameter or other maximum transverse dimension of the bonded perimeter may be less than or equal to 6.5 cm. In some embodiments, the diameter or other maximum transverse dimension of the bonded perimeter may be less than or equal to 6 cm. In some embodiments, the diameter or other maximum transverse dimension of the bonded perimeter may be less than or equal to 5 cm. Intermediate values between the foregoing are also contemplated, as are combinations of the foregoing and values greater than or less than the foregoing. For example, in some embodiments a diameter or other maximum transverse dimension of the bonded perimeter may be greater than or equal to 1 cm and less than or equal to 6.5 cm. In some embodiments, a diameter or other maximum transverse dimension of the bonded perimeter may be greater than or equal to 1.5 cm and less than or equal to 6.5 cm. Additionally, although diameters of bonded perimeters are described herein, it will be appreciated that the dimensions of the bonded perimeter described above are not limited to diameters, as the bonded perimeters are not limited to a circular geometry. For example, in a bonded perimeter formed in a rectilinear shape, the dimensions above may apply to a distance between opposing sides of the rectilinear shape.

In some embodiments, after bonding the membranes together (e.g., bonding of the perimeter and/or interior portions of the first membrane and the second), the first membrane and the second membrane may be coated with a hydrophilic material and/or subjected to other treatments which may not be compatible with the bonding process. In some embodiments, it may be desirable for one or more of the membranes included within a macroencapsulation device to be hydrophilic to facilitate loading of cells into the macroencapsulation device and/or to facilitate the flow of one or more fluids, biological compounds, therapeutics, cell nutrients, cell waste, and/or other materials through the membranes of a device. Additionally, a hydrophilic outer membrane may also reduce the occurrence of fibrosis when the device is positioned in vivo. Accordingly, the membranes of a macroencapsulation device may either be made from a hydrophilic material and/or treated with a hydrophilic coating. Appropriate hydrophilic materials may include, but are not limited to an appropriate hydrophilic polymer, polyethylene glycol, polyvinyl alcohol, polydopamine, any combination thereof, and/or any other appropriate hydrophilic material capable of forming a coating on the membranes or that the membranes may be made from.

In some embodiments, a root mean square (RMS) surface roughness of the bonded surface of a membrane and frame may be greater than or equal to 5 μm, 10 μm, 15 μm, 20 μm, 30 μm, and/or any other appropriate RMS surface roughness. The RMS surface roughness may also be less than or equal to 50 μm, 40 μm, 30 μm, 20 μm, 10 μm, and/or any other appropriate surface roughness. Combinations of the forgoing are contemplated including for example an RMS surface roughness that is between or equal to 5 μm and 50 μm. In some embodiments, the surface roughness may more preferably be between 5 μm and 10 μm. An RMS surface roughness value of a bonded surface may be measured using optical tomography based techniques, image analysis of cross sectioned bonded regions, and/or using any other appropriate technique.

While specific dimensions, parameters, and relationships related to the macroencapsulation device and the materials it is made from are described above, it should be understood that dimensions, parameters, and relationships both greater than and less than those noted above are contemplated as the disclosure is not limited in this fashion. Accordingly, any appropriate combination of size, construction, material properties, and/or relative performance parameters may be used for a device depending on the desired application.

Depending on the specific application and desired duration of use, a macroencapsulation device may be configured to have any appropriate fatigue life when implanted in vivo within a subject. For example, in some embodiments, a macroencapsulation device may be configured for implantation within the abdominal tissue of a subject where it may be subjected to abdominal contractions during use. Accordingly, in some embodiments, a fatigue life of a macroencapsulation device may be greater than or equal to 50,000 cycles, 60,000 cycles, 70,000 cycles, and/or 80,000 cycles. In some embodiments, a fatigue life of a macroencapsulation device may be greater than or equal to 50,000 cycles. In some embodiments, a fatigue life of a macroencapsulation device may be greater than or equal to 60,000 cycles. In some embodiments, a fatigue life of a macroencapsulation device may be greater than or equal to 70,000 cycles. In some embodiments, a fatigue life of a macroencapsulation device may be greater than or equal to 80,000 cycles. In some embodiments, the fatigue life may also be less than or equal to 1,000,000 cycles, 500,000 cycles, 200,000 cycles, 100,000 cycles, and/or 80,000 cycles. In some embodiments, the fatigue life may also be less than or equal to 1,000,000 cycles. In some embodiments, the fatigue life may also be less than or equal to 500,000 cycles. In some embodiments, the fatigue life may also be less than or equal to 200,000 cycles. In some embodiments, the fatigue life may also be less than or equal to 100,000 cycles. In some embodiments, the fatigue life may also be less than or equal to 80,000 cycles. Combinations of the foregoing ranges are contemplated including, for example, a fatigue life that is between or equal to 50,000 cycles and 200,000 cycles. Devices with a fatigue life both greater than and less than those noted above are also contemplated as the disclosure is not limited in this fashion. For the purposes of this application, a fatigue life of a macroencapsulation device may be determined using the fatigue cycle testing with a cyclically applied load of 12 N which is similar to the forces that may be experienced by a device when implanted in vivo within the abdominal tissue of a subject. In some embodiments, a fatigue life of the device using a cyclically applied load of 45 N measuring biphasic center mesh fatigue is between 1,000 cycles and 500,000 cycles. In some embodiments, a fatigue life of the device using a cyclically applied load of 45 N measuring biphasic center mesh fatigue is between 10,000 cycles and 80,000 cycles. In some embodiments, a fatigue life of the device using a cyclically applied load of 45 N measuring biphasic center mesh fatigue is between 40,000 cycles and 1,000,000 cycles.

In some embodiments, a cell population contained within a compartment of a macroencapsulation device may be an insulin secreting cell population. In some embodiments, a cell population contained within a compartment of a macroencapsulation device comprises a heterogeneous population of cells. In some embodiments, the cell population comprises at least one cell derived from a stem cell derived cell. In some embodiments, at least one cell is a genetically modified cell. In some cases, at least one cell is genetically engineered to reduce an immune response in a subject upon implantation of the device, as compared to comparable cells that are not genetically engineered. In some embodiments, the cell population is a stem cell derived cell that is capable of glucose-stimulated insulin secretion (GSIS). For example, an appropriate population of cells may comprise pancreatic progenitor cells, endocrine cells, beta cells, a matrix including one or more of the foregoing, or any combination thereof. Further, a matrix may comprise isolated islet cells, isolated cells from pancreas, isolated cells from a tissue, stem cells, stem cell-derived cells (e.g., stem cell-derived islet cells), induced pluripotent cells, differentiated cells, transformed cells, or expression systems, which can synthesize one or more biological products. In some embodiments, the macroencapsulation device comprises a population of stem cell-derived islet cells. In some embodiments, the stem cell-derived islet cells comprise stem cell-derived beta cells, stem cell-derived alpha cells, and/or stem cell-derived delta cells.

Depending on the particular embodiment, a therapeutically effective density of cells may be loaded into one or more compartments of a macroencapsulation device. Appropriate cell densities disposed within a compartment may be greater than or equal to about 1,000 cells/μL, 10,000 cells/μL, 50,000 cells/μL, 100,000 cells/μL, 500,000 cells/μL, 750,000 cells/μL, 1,000,000 cells/μL, and/or any other appropriate cell density. Appropriate cell densities disposed within the compartment may also be less than or equal to about 1,000,000 cells/μL, 500,000 cells/μL, 100,000 cells/μL, 50,000 cells/μL, 10,000 cells/μL, and/or any other appropriate cell density. Combinations of the foregoing are contemplated including cell densities between about 1000 cells/μL and 1,000,000 cells/μL. In some embodiments, cell densities disposed within the compartment is between 100,000 cells/μL and 1,000,000 cells/μL. In some embodiments, cell densities disposed within the compartment is between 75,000 cells/μL and 500,000 cells/μL. In some embodiments, cell densities disposed within the compartment is between 500,000 cells/μL and 1,000,000 cells/μL. In some embodiments, cell densities disposed within the compartment is between 750,000 cells/μL and 1,000,000 cells/μL. In some embodiments, cell densities disposed within the compartment is between 750,000 cells/μL and 1,250,000 cells/μL. Of course, cell densities both greater than and less than those noted above may also be used depending on the desired application and cell types being used.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

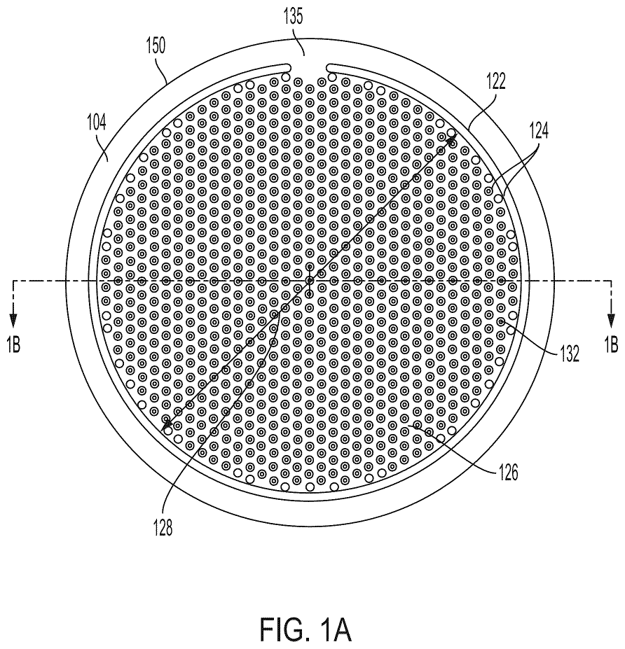

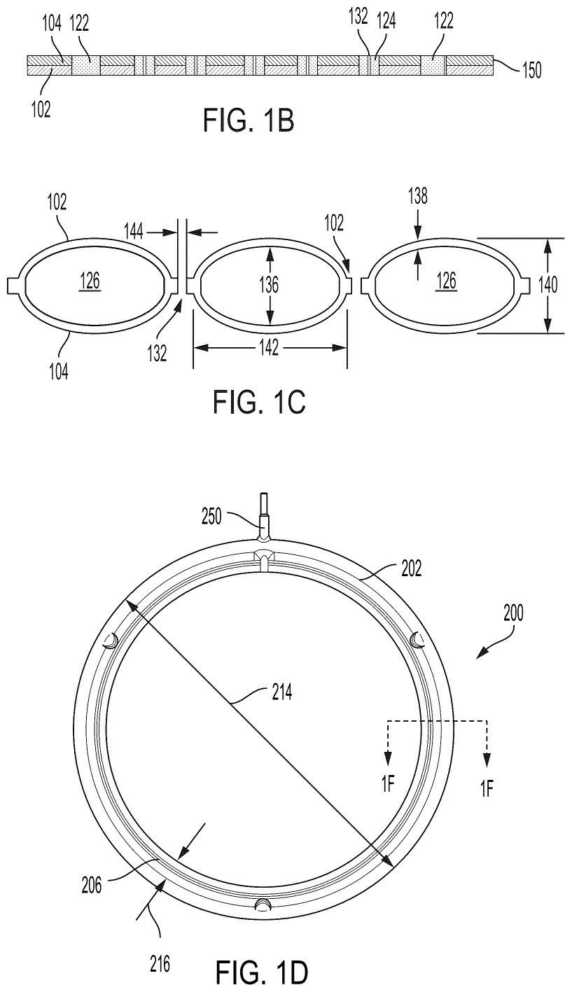

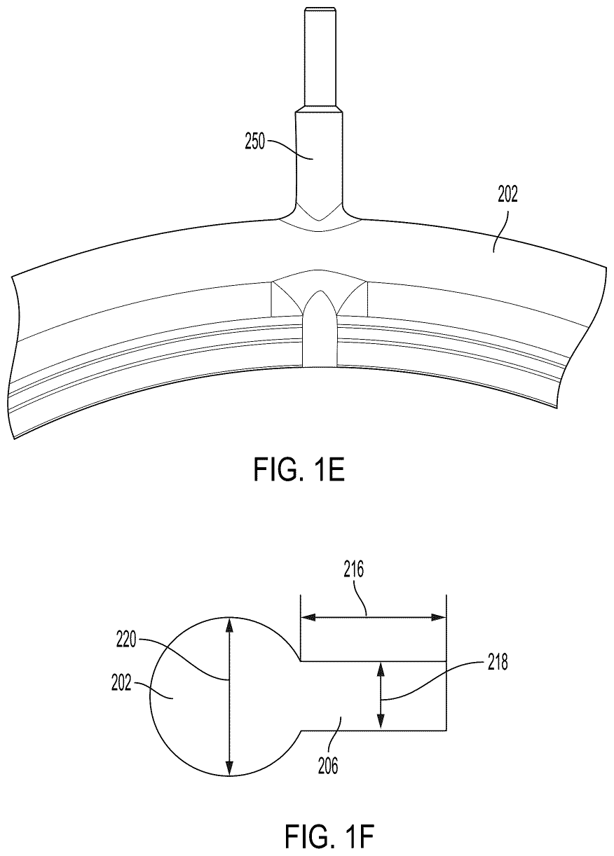

FIGS. 1A-1C show an embodiment of a membrane of a macroencapsulation device prior to mounting to a frame. As shown in the figures, a membrane may comprise a first membrane layer 102 and a second membrane layer 104. In some embodiments, the first and/or second membrane layer may comprise a polymer material, such as ePTFE. In various embodiments, each of the first membrane layer 102 and the second membrane layer 104 may be either sintered or unsintered. Each of the first and second membrane layers may also comprise a single layer or multiple layers.

The first and second membrane layers 102, 104 may be bonded together at a bonded perimeter 122 and bonded portions 124 located within the bonded perimeter. In FIG. 1A, a top surface of the second membrane layer 104 is shown with a bonded perimeter 122 of the membranes (e.g., where first and the second membrane layers are bonded) extending around a perimeter of the membranes. The bonded perimeter 122 may form an interior volume disposed between the first and second membrane layers configured to encapsulate a population of cells. In some embodiments, the bonded perimeter 122 may extend entirely around the perimeter of the membrane; however, as shown in FIG. 1A, the bonded perimeter may have an unbonded portion 135, for example to accommodate and/or cooperate with a fill port through which a population of cells may be introduced into an interior volume of the device. As will be appreciated, the dimensions of the bonded perimeter 122 may at least partially define a size of the interior volume. For example, in embodiments in which the membrane(s) and/or device have a generally circular shape, the bonded perimeter 122 may have a diameter 128, which may at least partially define a size of an interior volume between the first and second membrane layers.

In some embodiments, an interior volume between the first and second membrane layers may include a network of continuous interconnected volumes formed by and/or between the various bonded portions of the membrane. For example, as shown in FIG. 1C, an interior volume between the first membrane layer 102 and the second membrane layer 104 may include a network of volumes (e.g., channels 126) formed between the bonded portions 124. In some embodiments, an interior volume may have a volume thickness 136, which may be a maximum distance between the first and second membrane layers in a direction perpendicular to a maximum transverse dimension of the device. As will be appreciated, a total thickness of the membrane (e.g., the total thickness 140), may depend at least in part on the internal volume thickness 136, as well as the membrane layer thickness 138 for each membrane layer. As described herein, a thickness may be measured in a direction perpendicular to a maximum transverse dimension of the device.

As shown in the figures, the bonded perimeter may be disposed radially inward from the outer perimeter 150 of the membranes. The bonded portions 124 may take the form of bonded dots distributed across a surface area of the membranes in a hexagonal array. However, any appropriate shape, arrangement, configuration, and/or spacing of these bonded regions may also be used. For example, as shown in FIG. 1C, a bond spacing 142 may be a distance between adjacent bonded portions 124. Additionally, in some embodiments, one or more bonded portions 124 may include through holes 132 formed therein. Each through hole may have a through hole diameter 144. Due to the presence of these bonded regions located radially inwards from a bonded perimeter of the membranes, an internal volume formed between the membranes, once in the filled configuration (e.g., as shown in FIG. 1C), may take the form of a plurality of interconnected channels 126 corresponding to the unbonded regions of the membranes extending between these bonded portions.

In some embodiments, when the membrane layers are connected to a frame, the unbonded portion 135 may be positioned and sealed around a fill port of the frame such that the fill port remains in fluid communication with the interior volume. In some embodiments, a fill port may be included in the frame to allow fluid communication in at least one direction between an external environment and the interior volume of the device. For example, a fill port may be configured to allow the population of cells to be introduced into the volume between the first and second membrane layers. FIGS. 1D-1F illustrate one non-limiting example of a fill port 250 included on a frame 202 of a macroencapsulation device 200, according to one embodiment. The fill port includes a through hole, not depicted, extending through the fill port to an interior volume of the macroencapsulation device formed by the first and second membrane layers 102 and 104 shown in FIGS. 1A-1C above.

In various embodiments, a frame 202 may be formed in any appropriate shape, including any appropriate round, elongated, rectilinear, polygonal (e.g., pentagonal, hexagonal, octagonal, etc.), and/or any other appropriate regular or irregular shape. For example, in the embodiment shown, the frame 202 may be formed in a generally circular shape having a frame diameter 214, and/or a frame thickness 220. A frame thickness may be a maximum thickness between any two opposing surfaces or points of a cross-section of the frame. In some embodiments, the frame thickness 220 may be measured in a direction perpendicular to a maximum transverse dimension of the frame 202 and/or the device 200.

Additionally, in some embodiments, a device 200 may include a frame/membrane interface region 206, which may extend inwardly (e.g., radially inwardly) from the frame 202 and may be configured to be bonded with one or more membranes of the device. For example, as shown in the cross-sectional view of FIG. 1F (taken along line 1F-1F of FIG. 1D), the frame/membrane interface region 206 may extend from the frame 202 to provide one or more surfaces to which a membrane may be adhered, mechanically interlocked, fastened, secured, and/or bonded. The frame/membrane interface region may have an interface region width 216 where the frame and membrane(s) may be coextensive with one another, which may be a distance between an innermost portion of the frame 202 and an innermost portion of the frame/membrane interface region 206. Furthermore, the frame/membrane interface region may have an interface region thickness 218, which may be a thickness of the frame/membrane interface region in a direction perpendicular to a maximum transverse dimension of the frame 202 and/or device 200j.

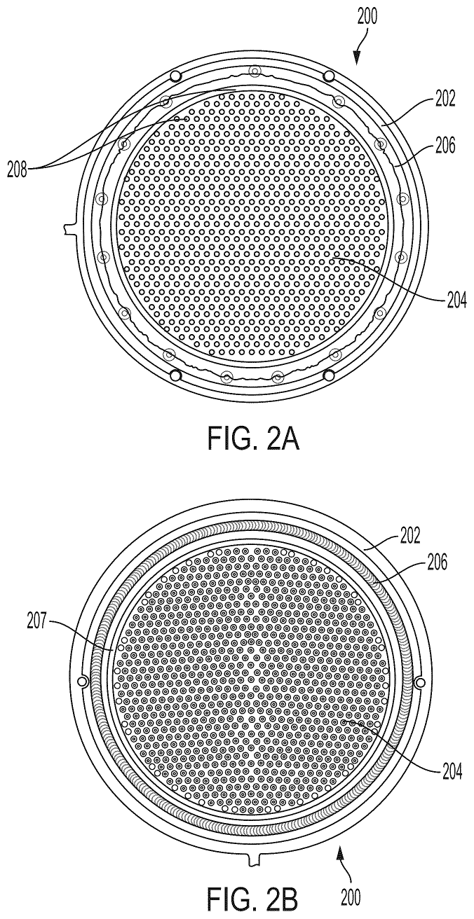

FIG. 2A and FIG. 2B each show a macroencapsulation device 200. Each macroencapsulation device 200 includes a frame 202, a membrane 204, and a frame/membrane interface region 206 bonded together using an adhesive (FIG. 2A) and with mechanically bonded regions (FIG. 2B). The frame/membrane interface region 206 as shown in the figures may be a region of the macroencapsulation device 200 where the membrane 204 and the frame 202 overlap one another. In some embodiments and as shown, the frame/membrane interface region 206 may be disposed around at least a portion of an outer perimeter of the membrane 204. For example, the frame/membrane interface region 206 may be disposed radially outward from a bonded perimeter 207 surrounding a sealed interior volume of the device. The membrane 204 and the frame 202 may be joined together at the frame/membrane interface region 206.

In some embodiments, a membrane may be mounted to a frame with slack in the membrane relative to the frame prior to being filled with a desired population of cells. When the membrane 204 is mounted to the frame 202, the slack may accumulate in certain areas along the interface between the frame and membrane. For example and as shown in FIG. 2A, pronounced membrane slack accumulations 208 may form during an adhesive mounting process. The slack accumulations 208 may be areas in which the membrane 204 bunches together or folds over itself. It will be appreciated that when the macroencapsulation device 200 flexes or bends, as may happen during use or implantation, stress concentrations may arise in the membrane 204 in areas including these slack accumulations 208 which may lead to a premature failure of the macroencapsulation device 200.

It may be noted that the macroencapsulation device 200 of FIG. 2B may have a more uniform distribution of slack and may not display any visible slack accumulations. As will be described below, the uniform distribution of slack in the membrane 204 may be achieved by the formation of mechanically interlocked regions at the frame/membrane interface region 206. In some embodiments, each mechanically interlocked region may include interlocks, overlaps, folds, or crimps in the membrane 204 which are mechanically locked within a plastically or thermoplastically deformed portion of the frame 202. In such embodiments, each mechanically interlocked region may take up a portion of the slack in the membrane 204 along the interface between the frame 202 and membrane 204. In embodiments where each mechanically interlocked region is substantially uniform in size, the substantially uniform mechanically interlocked regions may provide a substantially uniform distribution of slack along the frame/membrane interface region 206. Without wishing to be bound by theory, this may be due to deformation of the one or more membranes within the mechanically interlocked regions taking up at least a portion of the slack along the frame/membrane interface.

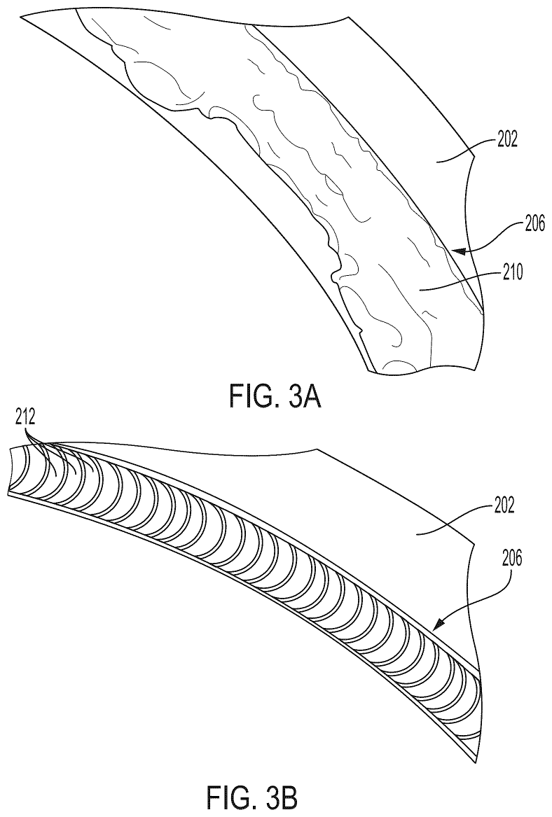

FIGS. 3A-3B each show a close-up of a frame/membrane interface region after a membrane has been removed from the frame 202. Because the membrane of each device has been removed, neither FIG. 3A nor FIG. 3B depicts a complete membrane. However, removal of the membrane from FIGS. 3A-3B allows for a clearer depiction of the frame/membrane interface region in each device. FIG. 3A shows a frame/membrane interface region 206 where the membrane had been joined to the frame 202 using an adhesive 210. FIG. 3B shows frame/membrane interface region 206 where a membrane had been joined to the frame 202 using a mechanical bonding technique as disclosed herein.

As shown in FIG. 3A, the adhesive 210 may be prone to non-uniform dispersion or distribution across the frame/membrane interface region 206. In some embodiments, this may lead to non-uniform strength in an adhesive bond between the membrane and the frame 202, resulting in a premature failure of the macroencapsulation device as described above.

As shown in FIG. 3B, a plurality of mechanically interlocked regions 212 may be formed at the frame/membrane interface region 206. Each mechanically interlocked region 212 may form a mechanically locking interface between the membrane and the frame. As illustrated in the figure, the mechanical bonding techniques disclosed herein may produce a more uniform bond at the frame/membrane interface region 206 both in terms of location, size, and strength as compared to the non-uniform adhesive bond shown in FIG. 3A.

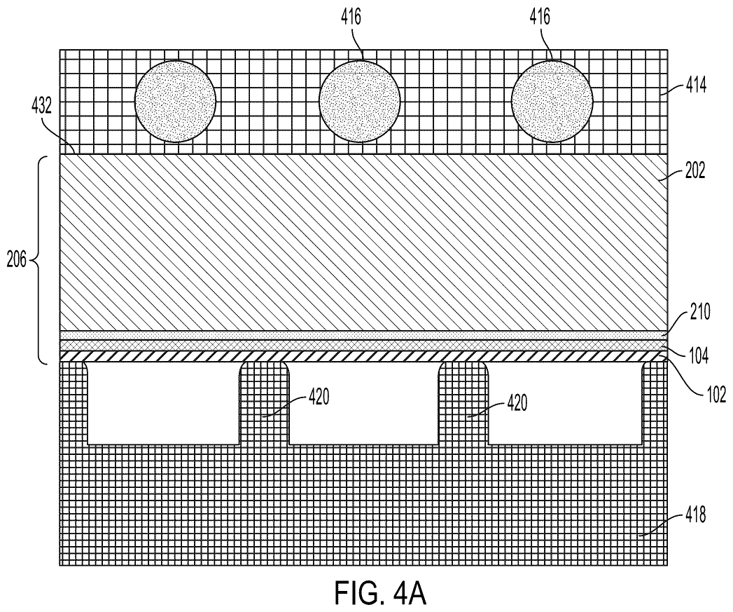

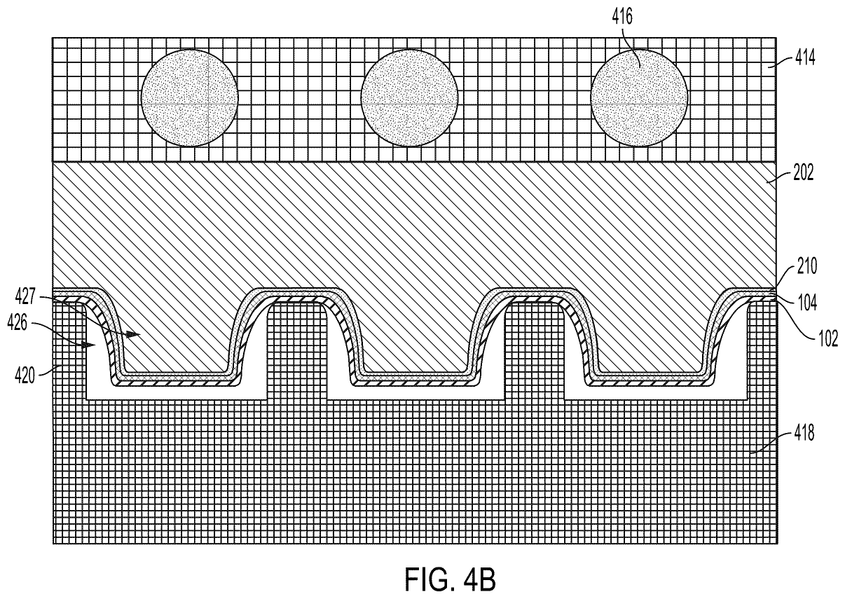

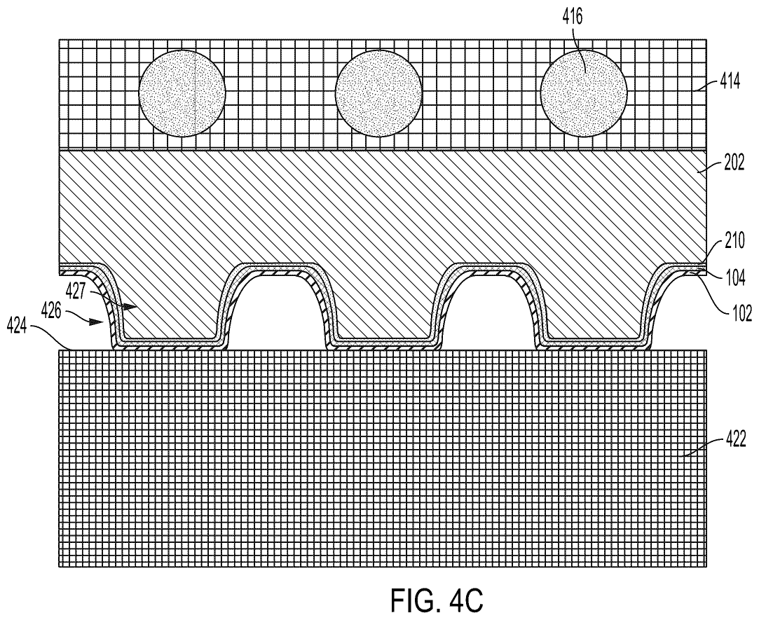

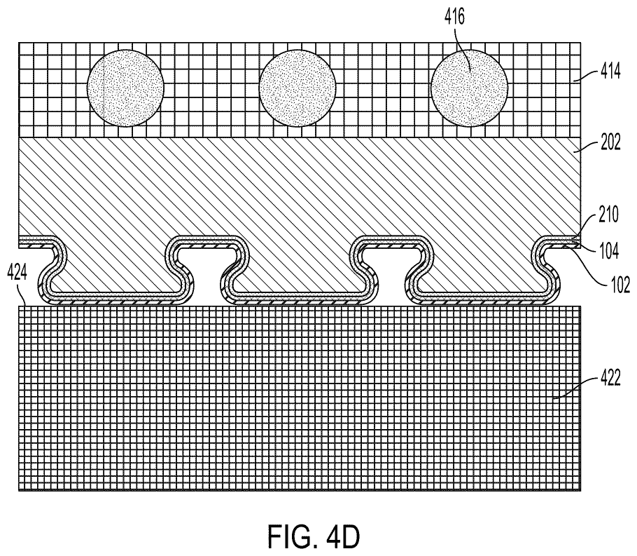

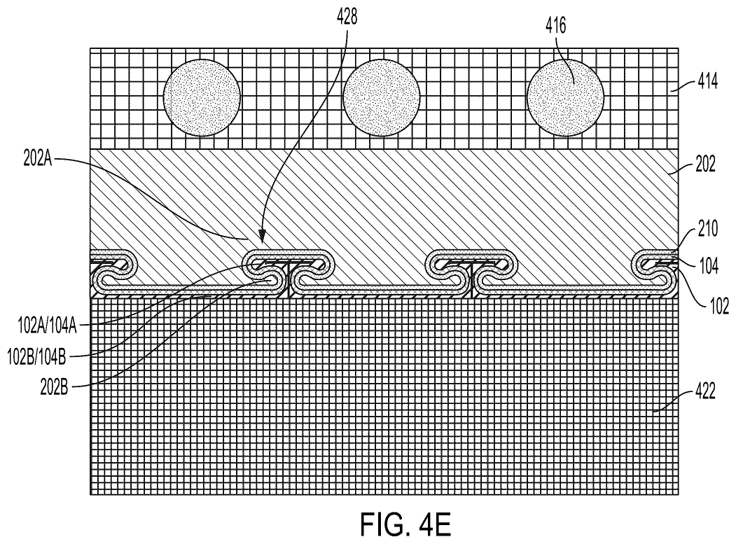

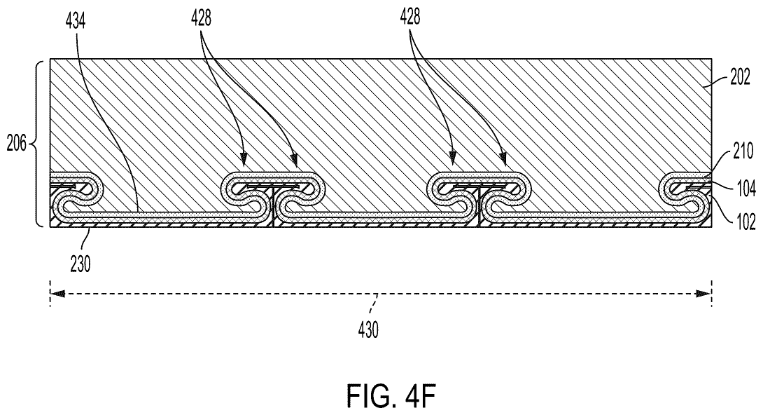

FIGS. 4A-4F depict a cross-section of a frame/membrane interface region 206 as might be seen along a portion of a length of the frame/membrane interface region of a macroencapsulation device. It will be appreciated that a plurality of mechanically interlocked regions may be formed as shown in FIGS. 4A-4F to extend around at least a portion of an outer perimeter of a membrane of the macroencapsulation device or along any other appropriately shaped interface between a membrane and frame. In some embodiments, the plurality of mechanically interlocked regions may form a sealed interior volume of the macroencapsulation device disposed between two opposing membrane layers of the macroencapsulation device. For example, the plurality of mechanically interlocked regions may form a compartment within which a population of cells may be encapsulated.

In FIG. 4A, a first membrane layer 102 and a second membrane layer 104 are overlaid on a frame 202. In some embodiments, an intermediate material layer 210 may be disposed between the one or more membranes of the device and the frame in. This intermediate material layer may include a material with a melting temperature that is between a melting temperature of the frame and the one or more membranes. Although the embodiment shown depicts a device having two membrane layers, it will again be appreciated that a macroencapsulation device as described herein may have any appropriate number of membrane layers as the disclosure is not limited in that regard.

In some embodiments of a method of manufacture, the first membrane layer 102, the second membrane layer 104, the frame 202, and the optional intermediate layer 210, may be placed between a first die 414 and a second die 418. In some embodiments, the first die 414 may comprise a supporting surface 432 which may be disposed against a corresponding surface of the frame 202. In the embodiment shown, the supporting surface 432 and the corresponding surface of the frame are depicted as flat for ease of illustration. However, it will be appreciated that a frame may be formed in any appropriate geometry, and that a die may be formed in any appropriate shape or configuration to correspond to or otherwise support the frame.

In some embodiments, the system may include a heater configured to heat the interface region between the membranes and frame. For example, the first die 414 may be associated with one or more heaters 416. The heaters 416 may be configured to provide heat to the frame 202 through the first die 414 or other appropriate portion of the system. In some embodiments, the heaters 416 may be one or more resistive heaters. In some embodiments, the heaters 416 may be one or more electric heater cartridges. In some embodiments, the heaters 416 may be one or more laser heaters. In some embodiments, the heaters 416 may be one or more radiation heaters. In some embodiments, the heaters 416 may be one or more inductive heater. In some embodiments, the heaters 416 may be one or more ultrasonic horns. Regardless of the specific heater used, the heaters 416 may heat the frame 202 to a temperature that is sufficient to facilitate plastic deformation of the frame 202, while maintaining a temperature of the membrane layers 102 and 104 at a temperature below a melting temperature and/or a sintering temperature of the membrane layers. In this respect, the frame 202 may be thermoplastically deformed without altering or compromising the material properties of portions of the one or more membranes adjacent to the bonded region.

In some embodiments, a second die 418 may also be positioned in the system such that the membrane layers 102 and 104 are disposed between the first die 202 and the second die. For example and as shown, the second die 418 may be placed in contact with the first membrane layer 102 on a side of the membrane layers opposite from the first die. The second die 418 may be configured to deform a portion of the flexible membrane layers and to thermoplastically deform an underlying portion of the frame. Deformation of the membrane layers and the frame may allow for the formation of one or more mechanically interlocking regions, as will be described below.

In some embodiments, formation of a plurality of mechanically interlocked regions may be facilitated by the formation of alternating raised and indented regions along a length of the membrane/frame interface. Accordingly, the second die 418 may include one or more crenellations 420 or other projections configured to form the desired indentations and raised regions. In the specifically depicted embodiment, each crenellation 420 may be configured to deform a portion of the membrane(s) and the frame when the crenellation 420 is pressed into the membrane and the frame at a desired forming temperature above a melting temperature of the frame. It will be appreciated that although the embodiment shown depicts a second die 418 having a plurality of crenellations 420 to form multiple deformations simultaneously, in other embodiments, a single crenellation, or set of crenellations, may be alternatingly pressed into and moved along a length of a membrane/frame interface to sequentially form different portions of the bonded interface. Additionally, it will be appreciated that the inclusion of crenellations only in the second die 418 of the depicted embodiment does not limit the placement of crenellations in other embodiments. For example, in some embodiments, crenellations may be included in the first die 414 either in addition, or as an alternative, to the crenellations shown on the second die 418.

FIG. 4B illustrates the second die 418 being moved relative to the first die 414 to compress and deform the first membrane layer 102, the second membrane layer 104, and the frame 202 between the dies. In embodiments in which the second die 418 includes crenellations 420, each crenellation 420, or other projection, may form a plurality of indentations 426 and raised regions 427 in the first membrane layer 102, the second membrane layer 104, and the frame 202 such that there are alternating raised regions and indented regions along at least a portion of a length of the interface. Again, this deformation may be done at a temperature where the frame is capable of being thermoplastically deformed and the one or more membranes maintain their structural integrity during the deformation process. For example, a temperature of at least a portion of the frame material and membrane may be greater than a glass transition temperature, and in some instances a melting temperature, of the frame material. The temperature may also be less than at least a melting temperature, and in some instances a sintering temperature or a glass transition temperature, of the one or more membranes. Again, this may allow the frame to be deformed without significantly altering material properties of the membrane within and/or adjacent to the bonded regions.

As shown in FIG. 4C, a third die 422 may also be used. In some embodiments, the second die 418 may be changed out for the third die 422. In some embodiments, after the second die 418 has completed a first deformation (for example, by creating indents 426 in the frame 202 and the membrane layers 102, 104 as shown), a third die 422 may be used to produce a second deformation. In some embodiments, the third die 422 may have a different geometry than the second die 418 in order to produce a second deformation that is different from a first deformation produced by the second die. For example, the third die 422 may include a flat surface 424 or other appropriately shaped surface that is configured to deform the raised regions 427 of the deformed membrane/frame interface.

As shown in FIG. 4D, the first die 414 and/or the third die 422 may be displaced to compress the frame 202 and the membrane layers 102, 104 therebetween. As the flat surface 424, or other appropriately shaped surface, is compressed against the raised portions of the frame 202 and the membrane layers 102, 104, the raised regions 427 may begin to fold into and on top of the indents 426 formed by the second die 418. Thus, the raised regions may be deformed to at least partially overlap the indented regions of the interface. It will be appreciated that the indents and raised regions may be deformed without forming voids because any air within an indent may be allowed to escape during the deformation process.

As shown in FIG. 4E, as the first die 414 and third die 422 continues to be compressed towards one another, the frame 202 and the membrane layers 102, 104 may be deformed such that portions of the membrane are folded over and compressed between the indents 426 and corresponding overlapping portions of the deformed raised regions to form a mechanically interlocked region 428. For example and as shown in the figure, a first portion 102A and 104A of the first and second membrane layers 102 and 104 may be compressed between a first portion 202A corresponding to an indented region of the frame 202 and a second portion 202B of the frame 202 corresponding to a raised region of the frame that at least partially overlaps the indented region. Due to its flexibility, the one or more membranes, e.g., membrane layers 102 and 104, extends out from and between these sequentially arranged mechanically interlocked regions.

It should be understood that in the above embodiments, the relative movement of the various dies may be coordinated in any appropriate manner. For example, the depicted dies in the various processing steps may be moved either together or individually to provide the desired relative movement to compress the frame and membranes therebetween

FIG. 4F depicts a frame/membrane interface region 206 of a macroencapsulation device after the frame and membrane have been bonded together. The interface region 206 may include a plurality of mechanically interlocked regions 428 as shown and described above. The interface region 206 may further include an exterior surface 230 of the one or more membranes that is substantially flat as a result of the second deformation by the third die 422 described above. However, embodiments, in which the exterior surface of the membranes located opposite from the frame is not flat along the membrane/interface region are also contemplated.

Comparing FIG. 4F to FIG. 4A, it will be appreciated that the mechanically interlocked regions 428 may facilitate the distribution of slack in the membrane layers 102, 104 throughout the frame/membrane interface region 206. Specifically, in each mechanically interlocked region 428, the membrane layers 102, 104 may be deformed from a flat configuration shown in FIG. 4A, to the interlocked configuration of FIG. 4F where portions of the flexible membranes have been folded into a T like shape such that a cross sectional length of the membrane in this region is less than a cross sectional length of the mechanically bonded region along the membrane/frame interface. Stated in another way, for a given linear length 430 along the frame/membrane interface region 206, a path length of an interface 434 between the membrane and the frame may be greater in the interlocked configuration than in the flat configuration. The interlocked configuration may therefore utilize a greater length of membrane than the flat configuration for the given linear length 430, thereby distributing the excess membrane slack throughout the frame/membrane interface.

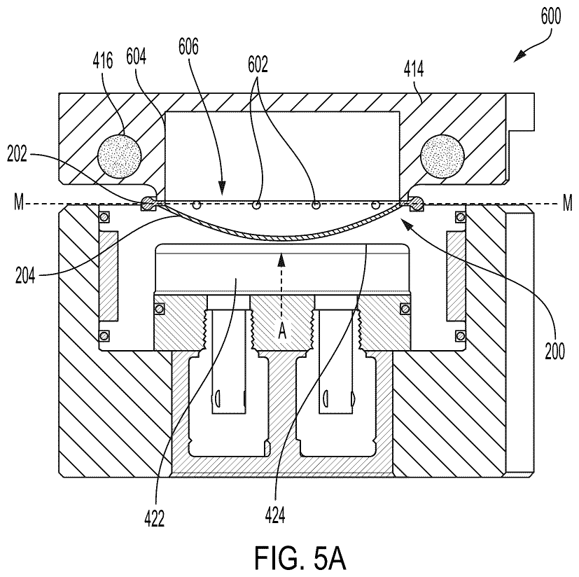

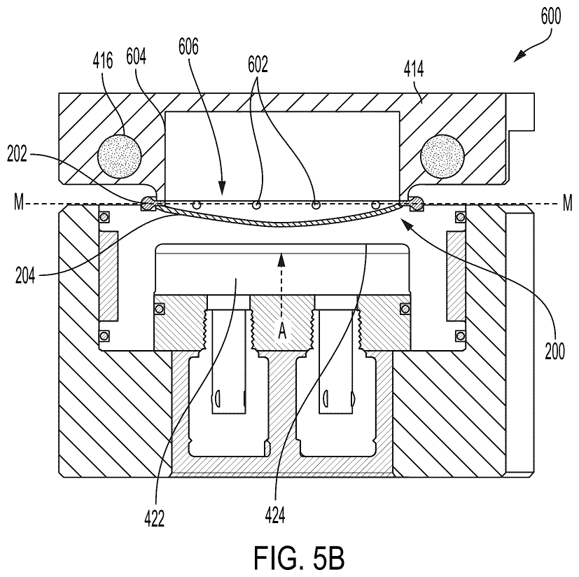

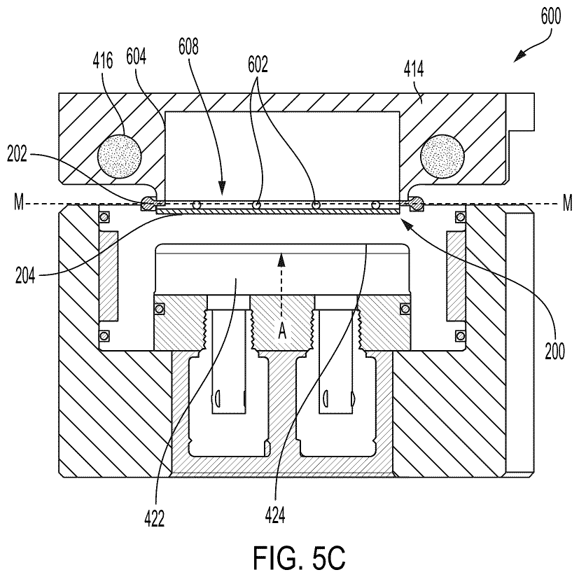

FIGS. 5A-5C depict schematics of various embodiments of a bonding apparatus 600 for manufacturing a macroencapsulation device 200 according to methods disclosed herein. As shown in each of FIGS. 5A-5C, a bonding apparatus 600 may comprise a plurality of dies, including a first die 414. The first die 414 may be configured to retain a frame 202 and/or one or more membranes 204 of the macroencapsulation device. In some embodiments, the first die 414 may include a retention mechanism configured to retain the frame 202 and/or the membrane(s) 204 against the first die. The first die 414 or the retention mechanism may be configured to retain the frame 202 and the membrane 204 in an overlapped configuration with at least a portion of the membrane 204 overlapping at least a portion of the frame 202 as shown. For example, in the depicted embodiment, the first die 414 may include a plurality of vacuum ports 602 for applying a retaining suction force to one or more portions of the macroencapsulation device 200. Each of the vacuum ports 602 may be coupled to a vacuum source (e.g., a pump, vacuum line, or other appropriate vacuum source). The vacuum ports 602 may be disposed at a location on the first die 414 corresponding to a desired retention location of the membrane 204, such that the first die 414 is configured to retain the membrane 204 in a desired position and configuration. In various embodiments, the suction force may be used to retain the membrane 204, the frame 202, or both the membrane and the frame. Although the depicted embodiment includes retention mechanisms comprising vacuum ports, it will be appreciated that some embodiments may additionally or alternatively include other retention mechanisms, including clips, detents, snap fittings, friction fittings, threaded arrangements, magnetic arrangements, fasteners, and/or any other appropriate retention mechanism.

The bonding apparatus 600 may further include one or more heaters 416. For example, the one or more heaters may be incorporated into the first die 414 or other appropriate portion of the bonding apparatus to apply heat to the frame. Specifically, the one or more heaters 416 may heat the first die 414 which may transfer the heat to the frame 202 to raise the temperature of the frame to a desired processing temperature. Of course, it should be understood that the one or more heaters may be incorporated into another portion of the bonding apparatus and/or may use any appropriate type of heating to heat the frame as the disclosure is not limited in this fashion.

In some applications, it may be desirable to maintain a temperature of an active portion of the one or more membranes (i.e., portions of the one or more membranes encapsulating the population of cells and through which material flows) below a temperature of the portion of the one or more adjacent portions of the membranes being bonding during formation of the plurality of mechanically interlocked regions. For example, in some embodiments, it may be desirable to prevent excessive heating of the active portions of the one or more membranes 204 of a macroencapsulation device, in order to substantially prevent property changes in the active portions of the one or more membranes. Therefore, in some embodiments, a bonding apparatus 600 may be configured to either cool and/or thermally insulate one or more portions of the one or more membranes 204 from the heat applied to the frame 202. For example, in the depicted embodiment, the first die 414 may include one or more heat shields 604 which may correspond to an insulating material, an air gap, or other appropriate insulating configuration disposed between the one or more heaters and active portions of the one or more membranes.

In one such embodiment, and as shown in the figure, the one or more heat shields 604 (e.g., the depicted air gap) may be positioned between a portion of the first die 414 against which the frame 202 is disposed during formation and a portion of the first die against which the membrane 204 is disposed during formation. As noted above, in other embodiments, active and/or passive coolers may be thermally coupled with one or more portions of the one or more membranes (e.g., an active portion of the one or more membranes) to remove heat from the one or more membranes during a formation process to maintain a temperature of the corresponding portions of the membrane below a desired operating temperature. Such coolers may include, but are not limited to, cooling channels formed in a structure, heat sinks, thermoelectric coolers, liquid cooling devices, forced ambient air cooling devices, forced chilled air cooling devices, chemical cooling devices (utilizing, for example, liquid nitrogen, dry ice, or other coolant chemicals), passive cooling device (utilizing, for example, cooling fins, thermally conductive materials, and/or specialized geometry), Peltier modules, and/or any other appropriate type of cooler. Of course, embodiments in which heat shields and/or coolers are not used to thermally isolate a portion of the one or more membranes are also contemplated.

In some embodiments, the first die 414 may optionally be configured to deform a portion of the membrane 204 to facilitate a uniform distribution of slack in the membrane throughout the frame/membrane interface. A plane of the membrane 204 may be defined when the membrane 204 is laid flat, as shown in FIG. 1B and as represented by line M-M in FIGS. 5A-5C. In some embodiments, a dome 606 may be included in the first die 414 to deform the membrane in a direction out of the plane of the membrane 204 and frame 202, see FIGS. 5A-5B. As will be appreciated by comparison of the embodiment of FIG. 5A with the embodiment of FIG. 5B, the dome 606, if included, may have any desired geometry or curvature, depending upon the embodiment. For example, increasing amounts of membrane slack associated with larger macroencapsulation volumes may be associated with larger domes and out of plane deformation of the one or more membrane layers during a bonding process, see the different dome sizes in FIGS. 5A and 5B. In some embodiments, the dome 606 of the different embodiments of FIGS. 5A-5C may include grooves, ridges, and/or crenellations as described above. In some embodiments, the dome 606 of the different embodiments of FIGS. 5A-5C may include grooves, such as radial grooves. In some embodiments, the dome 606 of the different embodiments of FIGS. 5A-5C may include ridges. In some embodiments, the dome 606 of the different embodiments of FIGS. 5A-5C may include crenellations. In still further embodiments, crenellations may be included on a clamp (not shown), which may be configured to cooperate with the bonding apparatus 600 or a die component thereof to produce a deformation in the macroencapsulation device.

In some embodiments, the first die 414 may not include a dome 606. For example, in the embodiment shown in FIG. 5C, the first die 414 may include a flat surface 608 instead of the dome shown in FIGS. 5A and 5B. In such embodiments, grooves, ridges, and/or crenellations may still be included on the first die 414. It will therefore be appreciated that the first die 414 may include any desired geometry, as the disclosure is not limited in this regard.

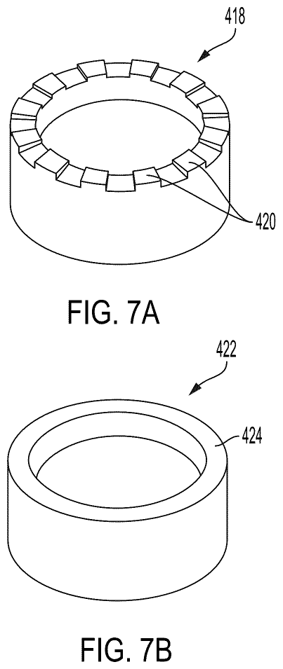

The plurality of dies of the bonding apparatus 600 may further include at least a second die (not shown; see FIGS. 4A-4B or FIG. 7A) and a third die 422. Each of the second die, the third die, and any further dies may be configured to produce a different deformation in the macroencapsulation device. For example, the second die may be configured to produce a first deformation (e.g., the indents 426 of FIGS. 4A-4C above), while the third die may be configured to produce a second deformation (e.g., the mechanically interlocking regions 428 of FIGS. 4E-4F above). In the embodiments of FIGS. 5A-5C, the third die 422 may include a flat surface 424 configured to produce the mechanically interlocking regions 428 as described above. The plurality of dies of the bonding apparatus 600 may be interchangeable within the bonding apparatus 600, such that a first deformation and a second deformation may be produced in a single macroencapsulation device 200 without needing to remove the macroencapsulation device 200 from the bonding apparatus 600. However, embodiments in which the separate formation processes are performed sequentially on different bonding apparatuses with different dies are also contemplated.

In operation, the macroencapsulation device 200 may be positioned against the first die 414 as shown. The vacuum source may be activated to retain the membrane 204 and the frame 202 against the first die 424. The heaters 416 may be activated in order to provide heat to the frame 202, while the heat shield 604 may prevent heat from the heaters 406 from excessively heating adjacent portions of the membrane 204 through the dome 606. The third die 422 may be pressed in the direction of arrow A in order to produce a desired deformation in the frame 202 and the membrane 204. Using either a single die or a plurality of dies as described above, a series of deformations may be produced, which may form a plurality of mechanically interlocked regions which secure the membrane 204 to the frame 202.

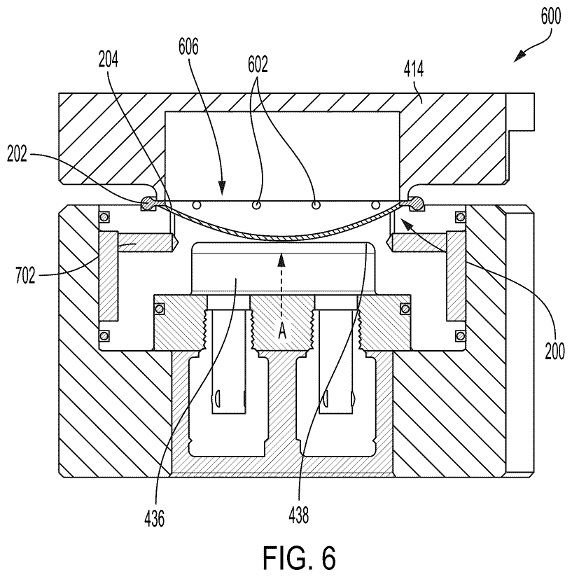

As part of the formation and bonding process, it may be desirable to seal adjacent membrane layers together to form a sealed interior cavity into which a population of cells may be introduced. FIG. 6 shows a modification of the bonding apparatus 600 for manufacturing a macroencapsulation device 200. This embodiment is substantially similar to the embodiments of FIGS. 5A-5C. However, a sealing die 436 may be configured to bond adjacent layers of the one or more membranes 204 in a region that is disposed radially inward from the frame 202. For example, such an embodiment may be used to form bonded portions, including a bonded perimeter, between layers of a membrane as described with respect to FIGS. 1A-1B, that extends at least partially around a perimeter of a sealed interior volume of the macroencapsulation device to form one or more compartments within the membrane 204 for housing a population of cells.

In the embodiment shown, a heater, such as the illustrated ultrasonic horns 702 or other appropriate heater, may be operatively coupled to the sealing die 436 and/or first die 414. During operation, the heater may heat the portion of the membrane compressed between the first die and the sealing die to facilitate bonding the layers of the one or more membranes 204 when the membrane layers are compressed between the first die and the sealing die. The use of ultrasonic horns in this embodiment may allow for the application of energy with sufficient precision to allow a bond to form in a desired area of the membrane 204 without compromising material properties of the membrane 204 in other areas. However, it will be appreciated that heat may be applied to bond the membrane layers using any appropriate heater.

FIGS. 7A-7B depict two embodiments of dies according to the present disclosure. In some embodiments, a die may be sized and shaped to accommodate a portion of corresponding die when pressed toward the corresponding die. For example, in the embodiments shown above, a first die 418 and a second die 422 may be sized and shaped to accommodate a dome or other geometry of a first die extending at least partially into the die, as shown in FIGS. 5A-6. In some embodiments, this may correspond to a die with a cylindrical tube-like shape with a channel extending at least partially through the die in an axial direction. A top surface of each die oriented towards the macroencapsulation device during formation may be configured to produce a desired deformation in a frame and/or a membrane of the macroencapsulation device. In some embodiments, the first die 418 may include a top surface having a plurality of crenellations 420 in order to produce a plurality of indents in a macroencapsulation device as described above, see FIG. 7A. In some embodiments, the second die 422 may include a flat surface 424 in order to further produce the mechanically interlocking regions as described above, see FIG. 7B.

While certain embodiments described herein contemplate the use of multiple dies, it will be appreciated that a desired deformation in a macroencapsulation device may be accomplished using only a single die, depending upon the desired deformation. For example, a position and/or orientation of a die (which may correspond to any structure used to deform the frame and membrane) may be controlled to produce the desired sets of deformations to produce a bond between the frame and one or more membranes.

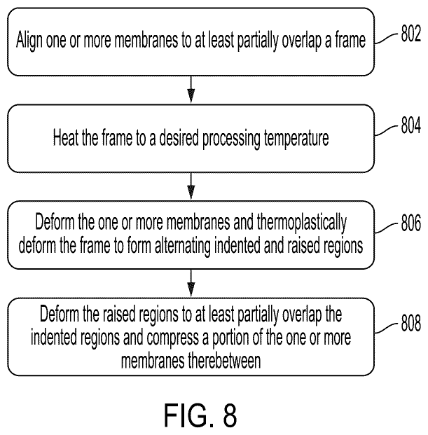

FIG. 8 depicts a flow chart showing one embodiment of a method of manufacturing a macroencapsulation device according to the present disclosure. At step 802, a membrane may be aligned to at least partially overlap the membrane with a corresponding portion of a frame of a macroencapsulation device. The overlapping portions of the one or more membranes and the frame may include a desired frame/membrane interface region to be bonded. At step 804, the macroencapsulation device or a portion thereof may be heated to facilitate deformation. For example, the frame of the macroencapsulation device may be directly, or indirectly heated, to a temperature that is greater than a glass transition temperature, and in some embodiments a melting temperature, of the frame. As noted previously, this temperature may be less than a melting temperature, and in some instances less than a sintering temperature, of the one or more membranes. At step 806, the one or more flexible membranes may be deformed, and the frame may be thermoplastically deformed, in order to form a plurality of alternating indented and raised regions along at least a portion, and in some instances the entire, length of the membrane/frame interface. The raised regions of the frame and one or more membranes may be deformed to at least partially overlap the indented regions and compress a portion of the one or more membranes between opposing portions of the deformed frame to form a plurality of mechanically interlocked regions disposed along a length of the membrane/frame interface.

### Example: Favorable Implanted and Explanted Durability Testing with Bond Interface Failure Mitigation and No Reduction in Cell Viability

As noted above, a strong, secure, and/or durable bond between a membrane and a frame may be achieved by the mechanically interlocking bonds described herein without significantly impacting the viability of surrounding tissue cells, or even with a positive impact on cell viability. For example, macroencapsulation devices manufactured using mechanically interlocking regions were implanted into minipigs alongside similar devices manufactured using adhesive bonds instead of the mechanical bonds. After a 6-month residence time, all devices were removed. It was observed that all devices remained intact, demonstrating the in vivo durability of the mechanically interlocking regions described herein. Viability of surrounding tissue cells was then determined, and each device was subjected to additional fatigue testing.

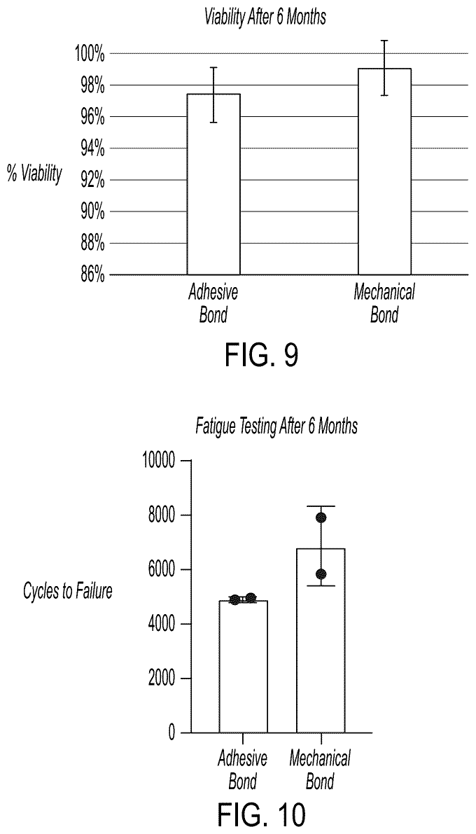

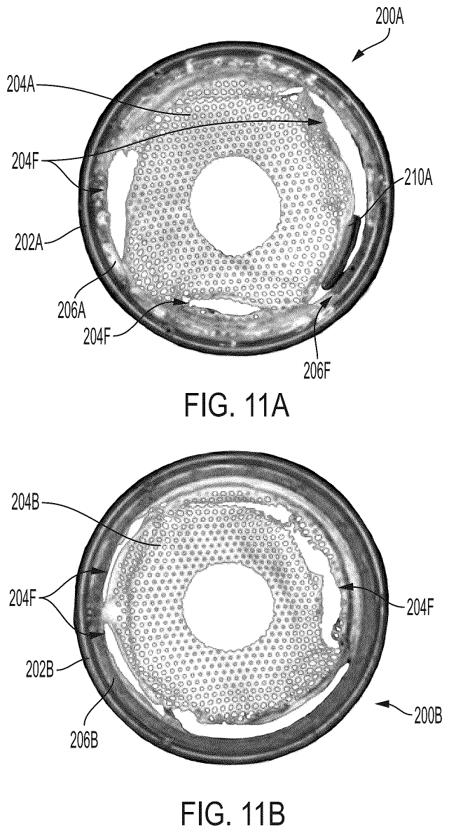

As shown in FIG. 9, cell viability for the mechanically bonded devices was similar to the viability of the adhesively bonded devices, with a slight improvement noted in the mechanically bonded devices. As shown in FIG. 10, the durability of the mechanically bonded devices was significantly improved over the adhesively bonded devices, with significantly higher cycles to failure observed. Moreover, with reference to FIGS. 11A-11B, failure in the mechanically bonded devices 200B was limited to the membranes 204B, as shown by membrane failures 204F. The mechanically interlocked interface between the membrane 204B and the frame 202B withstood the fatigue testing. By contrast, the adhesively bonded devices 200A failed both at the membrane 204A and at the interface region 206A between the membrane 204A and the frame 202A, as shown by membrane failures 204F and interface failures 206F, respectively. At the interface failures 206F, it was observed that the adhesive 210A had disbonded from the interface region 206A.

In view of the above, it will be appreciated that the mechanically interlocking regions described herein may significantly mitigate a failure mode which may be present in other bonding techniques. In particular, the mechanical interlocking bonds described herein may mitigate failures such as disbonds at an interface between a membrane and a frame. Furthermore, these benefits may be obtained without significant negative impact on the viability of cells within a device, or with a positive impact on cell viability.

### Example: Reduced Immune Response

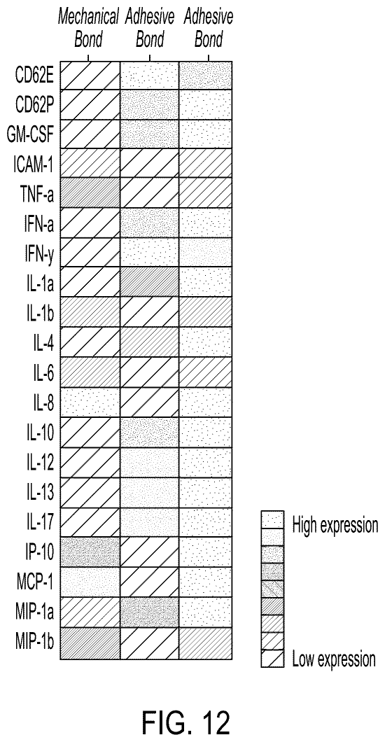

Further, as noted above, the mechanically interlocking regions described herein may result in a reduced immune response as compared to similar devices which are adhesively bonded. For example, mechanically bonded devices and adhesively bonded devices were each evaluated in a macrophage polarization assay to determine the levels of pro-inflammatory cytokine secretion provoked by each device. THP-1 macrophages were seeded on each device, and the levels of 20 different pro-inflammatory cytokines secreted by the macrophages were measured after 6 days. As shown in FIG. 12, reduced expression was observed in a majority of the cytokines in response to the mechanically bonded devices as compared to the adhesively bonded devices, with a substantial portion of cytokines showing a significantly reduced expression for the mechanically bonded devices. This indicates that the mechanically bonded devices result in a reduced immune response as compared to similar devices that are adhesively bonded.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of manufacturing a macroencapsulation device, the method comprising:
aligning one or more membranes of the macroencapsulation device with a frame of the macroencapsulation device such that a portion of the one or more membranes at least partially overlaps with a portion of the frame; and
deforming the portion of the one or more membranes and thermoplastically deforming the portion of the frame to form a plurality of mechanically interlocked regions of the one or more membranes and the frame.

2. The method of claim 1, wherein the plurality of mechanically interlocked regions extends around at least a portion of a perimeter of the one or more membranes.

3. The method of claim 1, wherein the plurality of mechanically interlocked regions extends at least partially around a sealed interior volume disposed between two layers of the one or more membranes.

4. The method of claim 3, wherein the sealed interior volume is configured to encapsulate a population of cells.

5. The method of claim 1, further comprising heating at least one of the one or more membranes and the frame.

6. The method of claim 1, wherein deforming the portion of the one or more membranes and thermoplastically deforming the portion of the frame comprises:
forming alternating indented and raised portions of the one or more membranes and the frame; and
deforming the raised portions of the one or more membranes and the frame to at least partially overlap the indented portions of the one or more membranes and the frame to form the plurality of mechanically interlocked regions.

7. The method of claim 1, wherein the one or more membranes comprise one or more materials selected from: polyvinylchloride (PVC), polyethylene (PE), polypropylene (PP), polymethylmethacrylate (PMMA), polystyrene (PS), polytetrafluoroethylene (PTFE), expanded polytetrafluoroethylene (ePTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly(lactic-co-glycolic acid) (PLGA), poly-L-lactide (PLLA), polyacrylonitrile (PAN), and electrospun PAN/PVC.

8. The method of claim 9, wherein at least one of the one or more membranes comprises ePTFE.

9. The method of claim 1, wherein the frame comprises a thermoplastic material.

10. The method of claim 9, wherein the frame comprises one or more materials selected from: polycarbonate, polyurethane, polyetheretherketone (PEEK), Polyvinyl Chloride (PVC), poly(oxymethylene), poly(methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), polycaprolactone, poly(lactide), poly(glycolic acid), poly lactide-co-glycolide, ethylene vinyl acetate copolymer, polyamides, poly(butylene) therephthalate, titanium, graphene, and stainless steel.

11. The method of claim 10, wherein the frame comprises polyetheretherketone (PEEK).

12. The method of claim 10, wherein the frame comprises fluorinated ethylene propylene (FEP).

13. The method of claim 1, wherein the one or more membranes include a first membrane and a second membrane disposed on the first membrane.

14. The method of claim 13, wherein the first membrane is sintered.

15. The method of claim 14, wherein the second membrane is unsintered.

16. The method of claim 14, wherein the second membrane is sintered.

17. The method of claim 1, wherein the method does not comprise applying an adhesive.

18. The method of claim 1, wherein the macroencapsulation device comprises a fill port.

19. A macroencapsulation device comprising:
one or more membranes including a sealed interior volume configured to encapsulate a population of cells;
a frame, wherein the one or more membranes are disposed on the frame; and
a plurality of mechanically interlocked regions of the one or more membranes and the frame extending around at least a portion of a perimeter of the one or more membranes.

20. The device of claim 19, wherein the plurality of interlocked regions includes alternating indented and raised portions of the one or more membranes and the frame, wherein the raised portions of the one or more membranes and the frame at least partially overlap the indented portions of the one or more membranes and the frame.

21. The device of claim 19, wherein the one or more membranes comprise one or more materials selected from: polyvinylchloride (PVC), polyethylene (PE), polypropylene (PP), polymethylmethacrylate (PMMA), polystyrene (PS), polytetrafluoroethylene (PTFE), expanded polytetrafluoroethylene (ePTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly(lactic-co-glycolic acid) (PLGA), poly-L-lactide (PLLA), polyacrylonitrile (PAN), and electrospun PAN/PVC.

22. The device of claim 21, wherein at least one of the one or more membranes comprises ePTFE.

23. The device of claim 19, wherein the frame comprises a thermoplastic material.

24. The device of claim 23, wherein the frame comprises one or more materials selected from: polycarbonate, polyurethane, polyetheretherketone (PEEK), Polyvinyl Chloride (PVC), poly(oxymethylene), poly(methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), polycaprolactone, poly(lactide), poly(glycolic acid), poly lactide-co-glycolide, ethylene vinyl acetate copolymer, polyamides, poly(butylene) therephthalate, titanium, graphene, and stainless steel.

25. The device of claim 24, wherein the frame comprises polyetheretherketone (PEEK).

26. The device of claim 24, wherein the frame comprises fluorinated ethylene propylene (FEP).

27. The device of claim 19, wherein the one or more membranes include a first membrane and a second membrane disposed on the first membrane.

28. The device of claim 27, wherein the first membrane is sintered.

29. The device of claim 28, wherein the second membrane is unsintered.

30. The device of claim 28, wherein the second membrane is sintered.

31. The device of claim 19, wherein the device does not comprise an adhesive.

32. The device of claim 19, wherein the device comprises a fill port.

33. A bonding apparatus for manufacturing a macroencapsulation device, the bonding apparatus comprising:
a retention mechanism configured to selectively retain a frame of the macroencapsulation device and one or more membranes of the macroencapsulation device in an overlapped configuration with at least a portion of the one or more membranes overlapping at least a portion of the frame;
a heater configured to heat at least the portion of the frame; and
one or more dies configured to deform the portion of the one or more membranes and thermoplastically deform the portion of the frame to form a plurality of mechanically interlocked regions of the one or more membranes and the frame extending around at least a portion of a perimeter of the one or more membranes.

34. The bonding apparatus of claim 33, wherein the retention mechanism is configured to retain the frame and the one or more membranes against a first die of the one or more dies.

35. The bonding apparatus of claim 34, wherein the first die is configured to deform an interior portion of the one or more membranes in an out of plane direction of the one or more membranes.

36. The bonding apparatus of claim 35, wherein the first die includes a dome configured to deform the interior portion of the one or more membranes in the out of plane direction.

37. The bonding apparatus of claim 36, wherein the dome includes radial grooves configured to distribute slack in the one or more membranes.

38. The bonding apparatus of claim 34, wherein the first die is configured to be coupled to a vacuum source to retain the one or more membranes against the first die via a suction force.

39. The bonding apparatus of claim 34, wherein the one or more dies includes a second die having a crenellated surface configured to deform the portion of the one or more membranes and thermoplastically deform the portion of the frame against the first die to form alternating indented and raised portions of the one or more membranes and the frame.

40. The bonding apparatus of claim 39, wherein the one or more dies includes a third die configured to deform the raised portions of the one or more membranes and the frame to at least partially overlap the indented portions of the one or more membranes and the frame to form the plurality of mechanically interlocked regions.

41. The bonding apparatus of claim 33, wherein the heater is at least one selected from a resistive heater, an ultrasonic horn, an electric heater cartridge, a laser heater, a radiation heater, or an inductive heater.

42. The bonding apparatus of claim 33, further comprising a heat shield and/or a cooler configured to maintain a temperature of an active portion of the one or more membranes below a temperature of the portion of the one or more membranes during formation of the plurality of mechanically interlocked regions.

43. The bonding apparatus of claim 33, wherein the plurality of mechanically interlocked regions forms a sealed interior volume.

44. The bonding apparatus of claim 43, wherein the sealed interior volume is configured to encapsulate a population of cells.
